(19) United States
(12) Patent Application Publication
(10) Pub. No.: US 2008/0299067 A1
(43) Pub. Date: Dec. 4, 2008

(54) Functionalised Siloxanes for Scar Tissue Treatment

(75) Inventors: Washington Sanchez, Capalaba (AU); Graeme Allan George, The Gap (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: QUEENSLAND UNIVERSITY OF TECHNOLOGY, QUEENSLAND (AU)

(21) Appl. No.: 11/579,378

(22) PCT Filed: May 4, 2005
(86) PCT No.: PCT/AU2005/00630

§ 371 Date: Nov. 2, 2006

Related U.S. Application Data
(60) Provisional application No. 60/568,109, filed on May 4, 2004.

(51) Int. Cl. A61K 31/765; A61L 27/60; A61P 17/02

(52) U.S. Cl. 424/78.06

(57) ABSTRACT

A novel composition for use in treating a wound, burn or other skin condition comprises one or more compounds of formula (I), wherein: m &equals; 0-6, n &equals; 6-100, Q, R and R′ may be independently selected from, C1-5 alkyl, OU, UOCH2CH3, CH2CH3, UOCH3, OH, O(CH2)y(OU)yCH3, (OCH2CH2)yOU, (OCH2CH2)yOH, UOH, UOU′, UCO2U′, CO2U, UCO2COU, CO2H, UCO2H, COX, UCOX, UCO2 R′, CO2COU, Aryl, ArylU, ArylUU, ArylUU′U″, NH2, UNH2, NHU, NUU′, NO2, UNO2, UCONH2, CONH2, UCONHU′, CONHU, UCONU′U″, CONU′U″, halogen, PO4H3, PO4H3-z, PO4H3-zU (z &equals; 0, 1, 2 or 3), PU3, P U′U″U′″SH, SO2 and SO3H; wherein U, U′, U″ and U′″ may be independently selected from any alkyl, alkenyl or alkynyl group where the number of carbon atoms is between 1 and 31; wherein X=halogen; wherein y=1-100; provided that Q, R and R′ can not all be C1 alkyl; and wherein the compound of formula (I) is present in an amount of at least 1% of the composition.

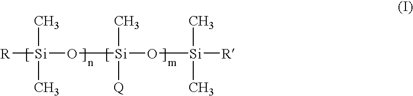

### FIELD OF THE INVENTION

The present invention broadly relates to a composition and method for the treatment of a wound, burn or other skin condition, without being limited thereto. In particular, the invention relates to a composition comprising one or more functionalised siloxanes which acts as an agent for treatment of skin conditions such as wounds, burns and scars, although without limitation thereto.

### BACKGROUND OF THE INVENTION

When skin or dermis has been wounded or traumatised by cutting or burning, scar tissue is formed. While all wounds heal by scar formation, in certain instances hypertrophic and/or keloid scars may form. Hypertrophic scarring results in erythematous, raised and thickened tissue due to overproduction of the extracellular matrix components. Keloid scarring results in raised formations of fibrous scar tissue, and like hypertrophic scarring, is caused by trauma and surgery. Such scarring is of particular concern in recovery from major burns injuries.

Conventional treatment of scarring utilises silicone polymers in the form of gel sheeting. Silicone polymers have been believed to be biologically inert and as such have enjoyed extensive use in various medical applications, eg. heart valve replacements and silicone in ‘hydrogel-based’ contact lens. They have also been used clinically to rehabilitate hypertrophic scarring, as an alternative to pressure therapy, X-ray therapy and corticosteroid injections. Silicone gels are virtually free of side effects. However, treatment is prolonged and requires up to twelve hours a day for up to several months (as described in, for eg. U.S. Pat. No. 6,337,076 B1 to SC Licensing Corporation). This prolonged scar treatment is inconvenient and investigations into more effective silicone gel treatment continue.

In attempting to understand the gel's efficacy, some proposed modes of action have included (i) increased rate of collagenase activity from increased skin temperature (ii) electrostatic induction (iii) skin hydration from occlusion and (iv) both chemical and mechanical effects of active components migrating from the gel. However, as discussed in U.S. Pat. No. 6,337,076, the mechanism of action remains unknown.

Medical grade silicone gels consist primarily of polydimethylsiloxane (PDMS), a synthetic polymer with the [(CH₃)₂SiO] repeating unit. Silicone gels are lightly cross-linked to form a three-dimensional matrix containing PDMS fluids. PDMS's have good biocompatibility, hydrophobicity and flexibility and find application in implants and pressure sensitive adhesives. Functionalized low molecular weight silicone fluids are used as enhancers in cosmetics (as described in JP2003081806 to Lion Corp) and pharmaceutical products for transdermal delivery agents (as described in U.S. Pat. No. 5,145,933 to Dow Corning). However, the reasons for their efficacy in scar remediation remain unknown.

### OBJECT OF THE INVENTION

Accordingly, it is an object of the invention to provide an efficacious and convenient composition and/or method for treating, wound, burn, scar tissue and/or other skin conditions which overcomes or alleviates one or more of the problems of the prior art or provides a useful commercial alternative.

### SUMMARY OF THE INVENTION

The present invention relates to a composition comprising a siloxane compound that is preferably capable of diffusing through the epidermis stratum corneum layer into the lower epidermis and dermis layer(s) and to thereby act as an efficacious treatment of wounds, burns, hypertrophic and keloid scar tissue reduction, and/or other skin conditions, without being limited thereto.

According to a first aspect of the invention there is provided a composition for use in treating a wound, burn or other skin condition comprising a compound of formula (I):

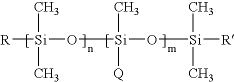

wherein:
   m=0-6
   n=6-100
   Q, R and R′ may be independently selected from, C₁₋₅ alkyl, OU, UOCH₂CH₃, CH₂CH₃, UOCH₃, OH, O(CH₂)_{y}(OU)_{y}CH₃, (OCH₂CH₂)_{y}OU, (OCH₂CH₂)_{y}OH, UOH, UOU′, UCO₂U′, CO₂U, UCO₂COU′, CO₂H, UCO₂H, COX, UCOX, UCO₂R′, CO₂COU, Aryl, ArylU, ArylUU′, ArylUU′U″, NH₂, UNH₂, NHU, NUU′, NO₂, UNO₂, UCONH₂, CONH₂, UCONHU′, CONHU, UCONU′U″, CONU′U″, halogen, PO₄H₃, PO₄H_{3-z}, PO₄H_{3-z}U (z=0, 1, 2 or 3), PU₃, P U′U″U′″ SH, SO₂ and SO₃H;
   wherein U, U′, U″ and U′″ may be independently selected from any alkyl, alkenyl or alkynyl group where the number of carbon atoms is between 1 and 31;
      wherein X halogen;
      wherein y=1-100;
      provided that Q, R and R′ cannot all be Cl alkyl; and
wherein the compound of formula (I) is present in an amount of at least 1% of the composition.

It will be appreciated that R and R′ may be terminating groups which join to form a bond or bridging alkyl group such that cyclic systems are formed.

According to an embodiment of the first aspect of the invention the compound of formula (I) is present in an amount of at least 5% of the composition.

According to another embodiment of the first aspect of the invention the compound of formula (I) is present in an amount of at least 10% of the composition.

According to yet another embodiment of the first aspect of the invention the compound of formula (I) is present in an amount of at least 30% of the composition.

According to a second aspect of the invention there is provided the use of a composition comprising a compound of formula (I):

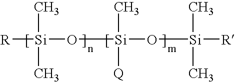

wherein:
   m=0-6
   n=6-100
   Q, R and R′ may be independently selected from, C₁₋₅ alkyl, OU, UOCH₂CH₃, CH₂CH₃, UOCH₃, OH, O(CH₂)_{y}(OU)_{y}CH₃, (OCH₂CH₂)_{y}OU, (OCH₂CH₂)_{y}OH, UOH, UOU′, UCO₂U′, CO₂U, UCO₂COU′, CO₂H, UCO₂H, COX, UCOX, UCO₂R′, CO₂COU, Aryl, ArylU, ArylUU′, ArylUU′U″, NH₂, UNH₂, NHU, NUU′, NO₂, UNO₂, UCONH₂, CONH₂, UCONHU′, CONHU, UCONU′U″, CONU′U″, halogen, PO₄H₃, PO₄H_{3-z}, PO₄H_{3-z}U (z=0, 1, 2 or 3), PU₃, P U′U″U′″SH, SO₂ and SO₃H;
   wherein U, U′, U″ and U′″ may be independently selected from any alkyl, alkenyl or alkynyl group where the number of carbon atoms is between 1 and 31;
      wherein X=halogen;
      wherein y=1-100;
         provided that Q, R and R′ can not all be C₁ alkyl;
         in the manufacture of a medicament for the treatment of a wound, burn or other skin condition, wherein the compound of formula (I) is present in the medicament in an amount of at least 1%.

According to a third aspect of the invention there is provided a method of treating a wound, burn or other skin condition including the step of administering to a patient a composition comprising an effective amount of a compound of formula (I):

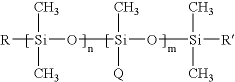

wherein:
   m=0-6
   n=6-100
   Q, R and R′ may be independently selected from, C₁₋₅ alkyl, OU, UOCH₂CH₃, CH₂CH₃, UOCH₃, OH, O(CH₂)_{y}(OU)_{y}CH₃, (OCH₂CH₂)_{y}OU, (OCH₂CH₂)_{y}OH, UOH, UOU′, UCO₂U′, CO₂U, UCO₂COU′, CO₂H, UCO₂H, COX, UCOX, UCO₂R′, CO₂COU, Aryl, ArylU, ArylUU′, ArylU′U″, NH₂, UNH₂, NHU, NUU′, NO₂, UNO₂, UCONH₂, CONH₂, UCONHU′, CONHU, UCONU′U″, CONU′U″, halogen, PO₄H₃, PO₄H₃ PO₄H_{3-z}U (z=0, 1, 2 or 3), PU₃, P U′U″U′″SH, SO₂ and SO₃H;
   wherein U, U′, U″ and U′″ may be independently selected from any alkyl, alkenyl or alkynyl group where the number of carbon atoms is between 1 and 31;
      wherein X=halogen;
      wherein y=1-100;
   provided that Q, R and R′ can not all be C₁ alkyl; and
      wherein the compound of formula (I) is present in an amount of at least 1% of the composition; and
      wherein said compound migrates through the stratum corneum layer to a lower epidermal skin layer.

According to an embodiment of the third aspect of the invention the method further includes the step of administering the composition topically.

According to another embodiment of the third aspect of the invention the method further includes the step of administering the composition in an amount effective to induce monocyte activation.

According to yet another embodiment of the third aspect of the invention the method further includes the step of administering the composition in an amount effective to suppress fibroblast growth.

According to a further embodiment of the third aspect of the invention the method further includes the step of administering the composition in an amount effective to down regulate collagen production.

According to a still further embodiment of the third aspect of the invention the method further includes the step of administering the composition in an amount effective to not inhibit cell proliferation.

According to a yet still further embodiment of the third aspect of the invention the method further includes the step of administering the composition in an amount effective to enhance collagenase activity.

In this specification, the terms “comprises”, “comprising” or similar terms are intended to mean a non-exclusive inclusion, such that a composition, method, system or apparatus that comprises a list of elements does not include those elements solely, but may well include other elements not listed.

### BRIEF DESCRIPTION OF THE DRAWINGS

In order that the present invention may be more readily understood and placed into practical effect, preferred embodiments of the invention will be described, by way of example only, with reference to the accompanying drawings and examples, in which:

TABLE 1: Molecular species extracted from Cica-Care® medical gel,

TABLE 2: Silicone diffusion across stratum corneum at different temperatures,

TABLE 3: List of functional groups substituents to PDMS's,

TABLE 4: List of functional groups substituents to PDMS's,

TABLE 5: List of functional groups substituents to PDMS's,

TABLE 6: Absorbance analysis of cells treated with siloxanes,

TABLE 7: Absorbance analysis at 540 nm using sirius red 80 of cells treated with siloxanes,

TABLE 8: Absorbance analysis at 540 nm using sirius red 80 of cells treated with siloxanes,

TABLE 9: Absorbance analysis at 540 nm using sirius red 80 of cells treated with siloxanes,

TABLE 10: Absorbance analysis at 540 nm using sirius red 80 of cells treated with siloxanes,

TABLE 11: Absorbance analysis at 540 nm using sirius red 80 of cells treated with siloxanes,

TABLE 12: Absorbance analysis at 540 nm using sirius red 80 of cells treated with siloxanes,

TABLE 13: Average results of foreskin fibroblasts and hypertrophic fibroblasts treated with siloxanes from microplate reader emission wavelength 540 nm sirius red in NaOH-Methanol 2.5 M,

FIG. 1a: MALDI-MS analysis of low molecular weight silicone oligomers from silicone medical gel after chloroform extraction. The progressions due to cyclic [symbol] methyl/methylol, methyl/methoxy-terminated (▪) and methyl/hydroxy-terminated oligomers () are shown,

FIG. 1b: Isotopic prediction of MALDI-MS analysis of low molecular weight silicone oligomers for: (A) NaSi₁₇C₃₄H₁₀₂O₁₇, (B) NaSi₁₇C₃₄H₁₀₂O₁₇SiC₂H₆CH₃OCH₃ and (C) NaSi₁₇C₃₄H₁₀₂O₁₇SiC₂H₆CH₃OH, maxima at n=17, from silicone medical gel after chloroform extraction,

FIG. 2: MALDI-MS analysis of low molecular weight silicone oligomers from silicone medical gel after water extraction. The spectrum shows contamination by polyethylene glycol at low molecular weight. The progressions due to cyclic (▾), methyl/methylol, methyl/methoxy-terminated (+) and methyl/hydroxy-terminated oligomers (♦) are shown,

FIG. 3: MALDI-MS of components transferred from fresh silicone medical gel by pressing against metal MALDI target. The spectrum shows contamination by polyethylene glycol at low molecular weight. The progression due to cyclic oligomers (▾) is shown,

FIG. 4: MALDI-MS of components transferred from water-washed silicone medical gel by pressing against metal MALDI target. The progression due to methyl/methylol-(methyl/methoxy)-terminated oligomers (▾) is shown,

FIG. 5: EDX elemental analysis of gelatine cross-section after 16 weeks in contact with Cica-Care® silicone gel: (a): Surface in contact with gel; (b): Bulk (centre); (c): Back surface; (d) Gelatine control,

FIG. 6: EDX elemental map for Silicon of gelatine cross-section after 16 weeks in contact with Cica-Care® silicone gel. Silicon appears as white zones against a dark background for different threshold sensitivities in (a) and (b),

FIG. 7: STEM image of cross-section of scar tissue (a) showing extent of epidermis (70 μm); (b) EDX map of Silicon showing maximum intensity at epidermis/dermis interface,

FIG. 8: MALDI mass spectrum from surface of scar tissue after contact with (chloroform) extract of Cica-Care® silicone gel showing the presence of cyclic (+) and methyl/hydroxyl-terminated () oligomers,

FIG. 9: In-vitro fibroblast cells incubated in the presence of low molecular weight functional silicones,

FIG. 10: In-vitro primary foreskin and hypertrophic derived fibroblast cells incubated in the presence of low molecular weight functional silicones (passage number indicated in brackets),

FIG. 11: Graphical representation of Table 7, foreskin fibroblast and hypertrophic derived fibroblast inoculated with functional silicones.

### DETAILED DESCRIPTION OF THE PREFERRED EMBODIMENTS

The invention provides a composition comprising one or more functionalised siloxanes such as set forth according to formula (I) which diffuse through the epidermis into the dermis layers and the use of the composition in the efficacious treatment of a wound, burn or other skin condition, including hypertrophic and keloid scarring.

As used herein, by “siloxane” is meant any of the large class of compounds that have alternate silicon and oxygen atoms.

As used herein; the term “functional group” or “functionalized” has its common definition, and refers to chemical moieties preferably selected from the group consisting of a halogen atom, C₁-C₁₅ alkyl, substituted C₁-C₁₅ alkyl, perhalogenated alkyl, cycloalkyl, substituted cycloalkyl, aryl, substituted aryl, benzyl, heteroaryl, substituted heteroaryl, cyano, and nitro. Functional groups may also be selected from the group consisting of —SRₛ, —OR_{O}, —NR_{Y1}R_{Y2}, —N⁺R_{q1}R_{q2}R_{q3}, —N═N—R_{q1}, —P⁺R_{q1}R_{q2}R_{q3}, —COR_{C}, —C(═NOR_{O})R_{C}, —CSR_{C}, —OCOR_{C}, —OCONR_{q1}R_{q2}, —OCO₂R_{C}, —CONR_{q1}R_{q2}, —C(═N)NR_{q1}R_{q2}, —CO₂R_{O}, SO₂NR_{q1}R_{q2}, —SO₃R_{O}, —SO₂R_{O}, —PO(OR_{O})₂, —NR_{q1}CSNR_{q2}R_{q3}. Substituents of these functional groups R_{q1}, R_{q2}, R_{q3}, R_{O} and R_{S} are preferably each separately selected from the group consisting of a hydrogen atom, C₁-C₁₅ alkyl, substituted C₁-C₁₅ alkyl, cycloalkyl, substituted cycloalkyl, aryl, substituted aryl, benzyl, heteroaryl, substituted heteroaryl and may constitute parts of an aliphatic or aromatic heterocyclic. R_{C} are preferably selected from the group consisting of a hydrogen atom, C₁-C₁₅ alkyl, substituted C₁-C₁₅ alkyl, perhalogenated alkyl, cycloalkyl, substituted cycloalkyl, aryl, substituted aryl, benzyl, heteroaryl, substituted heteroaryl and cyano.

As used herein, the term “alkyl” means any unbranched or branched, saturated hydrocarbon, with C₁-C₁₅ unbranched, saturated, unsubstituted hydrocarbons being preferred, and with methyl, ethyl, isobutyl, and tert-butyl being most preferred. Among the substituted saturated hydrocarbons, C₁-C₁₅, mono- and di- and pre-halogen substituted saturated hydrocarbons and amino-substituted hydrocarbons are preferred, with perfluoromethyl, perchloromethyl, perfluoro-tert-butyl, and perchloro-tert-butyl being the most preferred.

The term “substituted alkyl” means any unbranched or branched, substituted saturated hydrocarbon, with unbranched C₁-C₁₅ alkyl secondary amines, substituted C₁-C₁₅ secondary alkyl amines, and unbranched C₁-C₁₅ alkyl tertiary amines being within the definition of “substituted alkyl,” but not preferred. The term “substituted alkyl” means any unbranched or branched, substituted saturated hydrocarbon. Cyclic compounds, both cyclic hydrocarbons and cyclic compounds having heteroatoms, are within the meaning of “alkyl”.

As used herein, the term “alkenyl” means any unbranched or branched, substituted or unsubstituted, unsaturated hydrocarbon, with C₁-C₁₅ unbranched, mono-unsaturated and di-unsaturated, unsubstituted hydrocarbons being preferred, and mono-unsaturated, di-halogen substituted hydrocarbons being most preferred. The term “substituted alkenyl” means any unbranched or branched, substituted unsaturated hydrocarbon, substituted with one or more functional groups, with unbranched C₁-C₁₅ alkenyl secondary amines, substituted C₁-C₁₅ secondary alkenyl amines, and unbranched C₁-C₁₅ alkenyl tertiary amines being within the definition of “substituted alkyl”. The term “substituted alkenyl” means any unbranched or branched, substituted unsaturated hydrocarbon. Cyclic compounds, both unsaturated cyclic hydrocarbons and cyclic compounds having heteroatoms, are within the meaning of “alkenyl”.

As used herein, the term “alkynyl” means any unbranched or branched, substituted or unsubstituted, unsaturated hydrocarbon, with C₁-C₁₅ unbranched, mono-unsaturated and di-unsaturated, unsubstituted hydrocarbons being preferred, and mono-unsaturated, di-halogen substituted hydrocarbons being most preferred. The term “substituted alkynyl” means any unbranched or branched, substituted unsaturated hydrocarbon, substituted with one or more functional groups, with unbranched C₁-C₁₅ alkynyl secondary amines, substituted C₁-C₁₅ secondary alkynyl amines, and unbranched C₁-C₁₅ alkynyl tertiary amines being within the definition of “substituted alkyl”. The term “substituted alkynyl” means any unbranched or branched, substituted unsaturated hydrocarbon. Cyclic compounds, both unsaturated cyclic hydrocarbons and cyclic compounds having heteroatoms, are within the meaning of “alkynyl”.

As used herein, the terms “halo”, “halogen” and “halogen atom” refer to any one of the radio-stable atoms of column 17 of the Periodic Table of Elements, preferably fluorine, chlorine, bromine or iodine, with fluorine and chlorine being particularly preferred.

As used herein, the term “alcohol” means any unbranched or branched saturated or unsaturated alcohol, with C₁-C₆ unbranched, saturated, unsubstituted alcohols being preferred, and with methyl, ethyl, isobutyl, and tert-butyl alcohol being most preferred. Among the substituted, saturated alcohols, C₁-C₆ mono- and di-substituted saturated alcohols are preferred. The term “alcohol” includes substituted alkyl alcohols, and substituted alkenyl alcohols.

As used herein, the term “hydroxyalkyl” is preferably selected from a straight, branched, cyclic and bicyclic structures and combinations thereof, having 1 to 15 carbon atoms, substituted with one or more hydroxyl groups. Suitable hydroxylalkyls may be selected from hydroxymethyl, hydroxyethyl, hydroxypropyl and hydroxybutyl.

As used herein, the term “aryl” or “Ar” encompasses the terms “substituted aryl,” “heteroaryl,” and “substituted heteroaryl” which refers to aromatic hydrocarbon rings, preferably having five or six atoms comprising the ring. The terms “heteroaryl” and “substituted heteroaryl” refer to aromatic hydrocarbon rings in which at least one heteroatom, for example, oxygen, sulphur, or nitrogen atom, is in the ring along with at least one carbon atom. “Aryl,” most generally, and “substituted aryl,” “heteroaryl,” and “substituted heteroaryl” more particularly, refer to aromatic hydrocarbon rings, preferably having five or six atoms, and most preferably having six atoms comprising the ring. The term “substituted aryl” includes mono and poly-substituted aryls, substituted with, for example, alkyl, aryl, alkoxy, azide, amine, and amino groups. “Heteroaryl” and “substituted heteroaryl,” if used separately, specifically refer to aromatic hydrocarbon rings in which at least one heteroatom, for example, oxygen, sulfur, or nitrogen atom, is in the ring along with at least one carbon atom.

In a preferred embodiment the invention provides a composition for use in treating a wound, burn or other skin condition comprising a compound of formula (I):

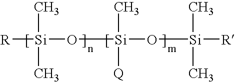

wherein:
   m=0-6
   n=6-100
   Q, R and R′ may be independently selected from, C₁₋₅ alkyl, OU, UOCH₂CH₃, CH₂CH₃, UOCH₃, OH, O(CH₂)_{y}(OU)_{y}CH₃, (OCH₂CH₂)_{y}OU, (OCH₂CH₂)_{y}OH, UOH, CO₂U, CO₂H, UCO₂H, UCO₂R′, CO₂COU, Aryl and ArylU;
   wherein U is selected from any alkyl, alkenyl or alkynyl group where the number of carbon atoms is between 1 and 31;
      wherein y=1-100;
      provided that Q, R and R′ can not all be C1 alkyl; and
wherein the compound of formula (I) is present in an amount of at least 1% of the composition.

In another preferred embodiment the invention provides a composition for use in treating a wound, burn or other skin condition comprising a compound of formula (I):

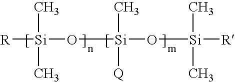

wherein:
   m=0-6
   n=6-100
   Q, R and R′ may be independently selected from, C₁₋₅ alkyl, OH, O(CH₂)_{y}(OU)_{y}CH₃, (OCH₂CH₂)_{y}OU and UOH;
   wherein U is selected from any alkyl, alkenyl or alkynyl group where the number of carbon atoms is between 1 and 31;
      where in y=1-100;
      provided that Q, R and R′ can not all be C1 alkyl; and
wherein the compound of formula (I) is present in an amount of at least 1% of the composition.

In particular embodiments when m is present in formula (I) m is equal to 1, 2, 3, 4, 5 or 6.

In other particular embodiments, n in formula (I) is has a value in the range 10 to 50, 20 to 30 or any integer value between 10 and 50.

In particular embodiments, the number of carbon atoms in U of formula (I) is any integer value in the range 1 to 10.

In other particular embodiments the number of carbon atoms in U of formula (I) is an integer value in the range 2 to 6.

In particular embodiments y in formula (I) is any integer value between 1 and 100.

In even more particular embodiments, the value of y may be 5, 10, 15, 20, 25, 30, 35, 40, 45, 40, 55, 60, 65, 70, 75, 80, 85, 90, 95 or 100.

In a particular embodiment the compound of formula (I) may be an ethoxy silicone compound, a methoxy silicone compound, or a compound hereinafter referred to as GP582, GP426, PG507, GP226 or GP218.

In particular preferred embodiment the compound of formula (I) is GP507, GP226 or GP218.

In particular embodiments the siloxane may constitute at least 5%, 10%, 15%, 20%, 25%, 30%, 40%, 45%, 50%, 55%, 60%, 65%, 70%, 75%, 80%, 95% of the composition.

Preferably the siloxane may be at a concentration in the range 15%-45%, More preferably the range is about 30-35%.

It is understood that as used in the specification and the claims appended hereto the terms amount and concentration are used interchangeably and have the same meaning.

The composition and/or method of treatment may be suitable for any animal, inclusive of mammals such as humans, domestic animals, performance animals and livestock.

Preferably, the mammal is a human.

As previously described the invention relates to the administration of one or more of the siloxane compounds as a pharmaceutical composition.

As hereinbefore described the composition may be used according to the third aspect of the invention.

Suitably, the composition further comprises a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

Preferably, the pharmaceutically-acceptable carrier, diluent or excipient is suitable for administration to mammals, and more preferably, to humans.

In one embodiment, the pharmaceutical composition may be a dermatological composition comprising a dermatologically-acceptable carrier, diluent or excipient.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

In a particular embodiment the composition is suitable for topical administration.

In another particular embodiment the composition for topical administration is a cream, unguent or lotion.

In another particular embodiment the composition comprises one or more of the group consisting of the ethoxy silicone, the methoxy silicone, GP582, GP426, GP507, GP226 and GP218.

In still another particular embodiment the composition for topical administration comprises one or more of the group consisting of GP218, GP226 and GP507.

Preferably the composition is administered in a manner in which the silicone migrates through the layers of the skin and/or other tissues.

The inventors envisage including in the formulation a pigment to mask the redness of highly vascularized scarring on prominent facial and body parts.

The formulation may also include a sunscreen agent, an anaesthetic agent or other conventional and known additives for formulations applied to skin.

It is understood that silicones to be used in therapeutic or cosmetic formulation must not contain any silanol or any other functional groups that render them allergenic or irritant.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be affected by using other polymer matrices, liposomes and/or microspheres.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner. It is understood that a person of skill in the art is readily able to determine appropriate dosage for a patient.

It is understood that the compositions and methods of the invention can form part of a kit, or form part of utilizing a kit. The person of skill in the art readily understands how to construct the kit based on the information contained herein and common general knowledge.

The term “pharmaceutically acceptable salt,” especially when referring to a pharmaceutically acceptable salt of the compound of Formula (I), refers to any pharmaceutically acceptable salts of a compound, and preferably refers to an acid addition salt of a compound. A preferred example of a pharmaceutically acceptable salt is an acid addition salt of a compound. Other preferred examples of a pharmaceutically acceptable salt are the alkali metal salts (sodium or potassium), the alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane. With respect to compounds of the invention that are basic amines, the preferred examples of pharmaceutically acceptable salts are acid addition salts of pharmaceutically acceptable inorganic or organic acids, for example, hydrohalic, sulfuric, phosphoric acid or aliphatic or aromatic carboxylic or sulfonic acid, for example acetic, succinic, lactic, malic, tartaric, citric, ascorbic, nicotinic, methanesulfonic, p-toluensulfonic or naphthalenesulfonic acid. Preferred pharmaceutical compositions of the present invention include pharmaceutically acceptable salts of the compound of Formula (I).

The term “erythema” means any skin redness, especially a chronic skin redness having a neurogenic origin.

The term “sodiated ion” means an ion formed by the binding of a cation to a neutral molecule, such as those formed by the binding of Na or K to neutral molecules: [M+Na]⁺ or [M+K]⁺.

So that the invention may be fully understood and put into practical effect, the invention is described with reference to the following non-limiting examples.

### EXAMPLES

### Experimental

### Example 1

### Identification of Migrating Low Molecular Weight Species from Cica-Care® Silicone Gel

The silicone gel Cica-Care® (Smith and Nephew) was obtained from the Burns Unit, Royal Brisbane Hospital. MALDI-TOF-MS has been used to determine the chemical composition, molar mass and oligomeric distribution of the species present in both the bulk and those that may migrate from the surface of the silicone gel.

In the studies to determine the composition of low molecular weight species present in the bulk, the gel was exhaustively extracted with chloroform. This resulted in a loss in weight of 36% from the cross-linked gel.

In another experiment the gel was extracted with water to determine if any water-soluble or hydrophilic species were present in the gel. The weight loss was much less (4%).

For the analysis of surface species, transfer to the MALDI target was achieved by touching the surface of the gel to the stainless steel plate, which formed the MALDI target. To prepare the sample for MALDI analysis, typically 1 pg of sample was added to the matrix (20:1 of 4-hydroxybenzilidene malononitrile: sodium iodide) on the target plate and allowed to dry. The ions from 450 N₂ laser shots at 337 nm were analysed in a Micromass T of-Spec 2E mass spectrometer.

GC/MS was used to detect any lower molecular mass species below 960 Da that were present in the extracts. Samples were prepared by separate extractions of the silicone gel with methanol. These were later analysed with a Fisons 8000 gas chromatograph with a HT5 capillary column combined with a quadrupole MD800 mass spectrometer for structural analysis. The 12 m column has a 5% phenyl (equiv.) polycarborane-siloxane with an internal diameter of 0.22 mm. All injections were made in split-less mode with a purge activation time of 60s. During the purge activation, the column temperature was held at 40° C. after which the temperature was ramped to 350° C. at rate of 15° C./min.

### Example 2

### Results and Discussion of Isolation of Dominant Species

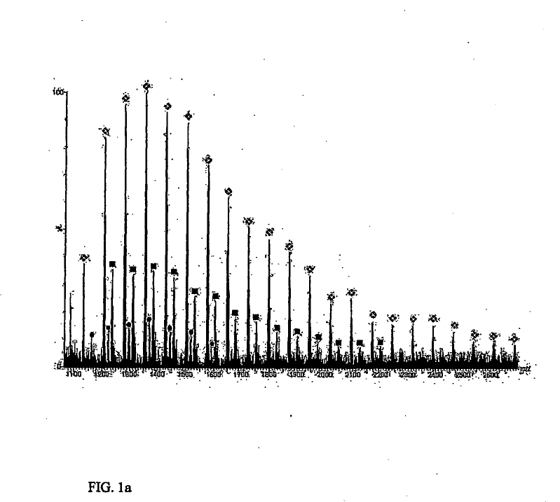

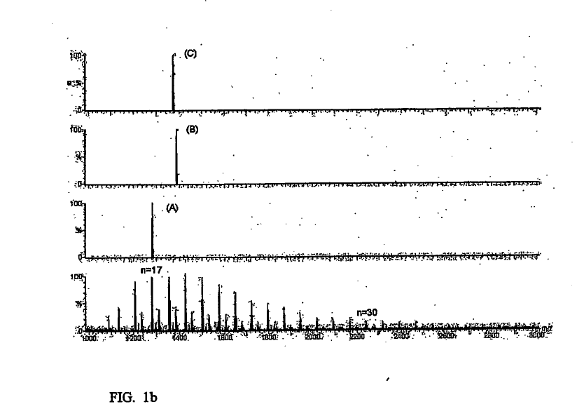

FIG. 1 shows the MALDI mass spectrum of the low molecular weight silicone species obtained by extracting the gel with chloroform. The spectrum shows the species present from mass 1080 Da to 2640 Da. The cut off of 1080 Da is set to ensure that interference from adduct ions of the matrix (n×192 Da) is eliminated in the analyte spectrum.

In FIG. 1a the progression of peaks with a separation of 74 Da corresponding to [(CH₃)₂—Si—O] is the dominant feature. The peaks correspond to the sodiated species for increasing values of n, with a peak at around n=17. Such species are normally not detected by analytical methods such as GC/MS due to mass limitations and reflects the sensitivity of MALDI-MS. It is recognised however that poly-disperse samples show selective ionisation and detection so that results from un-fractionated samples may be biased to lower molar masses (Montaudo, et al., Rapid. Commun, Mass Spectrom., 1995, 9, 1158). The sodiated species in the MALDI spectra are observed due to the presence of sodium iodide in the matrix to aid ionization. The two most likely species present in a polydimethylsiloxane sample are the linear (a) and cyclic (b) oligomers as shown

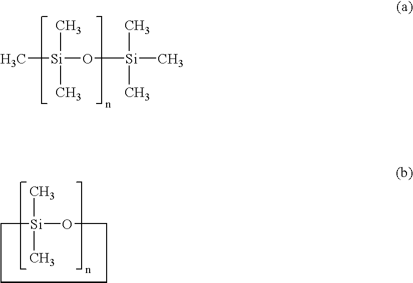

Since PDMS is synthesised by an equilibrium of ring opening polymerisation of the cyclic starting material with n=4 (octamethyl cyclotetrasiloxane or D4) there will always be cyclic oligomers at equilibrium. Thus ˜10% of the silicone is present as low molecular weight cyclic oligomers in the PDMS in addition to the linear oligomer species. Using the mass spectrometer's instrumental software it is possible to simulate the isotopic species to be expected for the linear and cyclic oligomers for n=17 (Hunt, et al., Polym. Int., 2000, 49(7), 633). When this is performed, (as shown in FIG. 1(b)) the spectrum may be assigned to the cyclic species, with a smaller contribution from linear species which are not those with methyl functionalities at each end (as shown in (a) above) but rather those having one end group either a methoxy or a methylol group.

Simulations have been performed for all possible terminal groups in addition to the methyl/hydroxyl-, methyl/methylol- and methyl/methoxy combination shown in FIG. 1, but no further improvement to fit of the data was obtained. The combinations of end group trialled were hydrogen/hydrogen, methyl/hydrogen, methyl/methyl, methyl/hydroxyl, hydroxyl/hydroxyl, methoxy/methoxy, methylol/methylol, methyl/vinyl, silanol/vinyl and vinyl/vinyl.

The inventors thereby conclude that extraction of the silicone gels with chloroform removes all species both cyclic and linear but the detection by MALDI of the cyclic species may be favoured due to the ionization process (Axelsson, et al. Macromolecules, 1996, 29, 8875-8882).

The species below 960 Da have been determined by GC/MS. This resulted in the positive identification of peaks corresponding to D5 and D6. The absence of D4 is consistent with the stripping of this volatile species from the gel.

### Example 3

### Model Collagen System for Assessing Migratory Silicone Species

Gelatine, or hydrolysed bovine collagen, was used as a model system to determine the migration of silicone species from the medical gel. Gelatine was cast from solution containing an antibiotic to prevent microbial attack during the trial. The silicone gel was placed in contact with the gelatine surface for 16 weeks and then sectioned after freeze-drying to obtain cross-sections for elemental mapping of silicon by Energy Dispersive X-Ray (EDX) analysis in the Scanning Electron Microscope. Scanning Transmission Electron Microscopy (STEM) has also been performed on sectioned scar tissue. Samples previously stored under liquid nitrogen were cryo-sectioned to 1-2 μm by 1 mm² with an RMCTVII ultramicrotome at liquid nitrogen temperatures using a glass knife. The cryo-sections were then collected on double grids and immediately freeze-dried under vacuum (104 mmHg) overnight. X-ray microanalyses were performed with a Philips CM200 scanning transmission electron microscope at 120 kV and a silicon map constructed.

### Example 4

### MALDI Analysis of Aqueous Gel Extraction Constituents

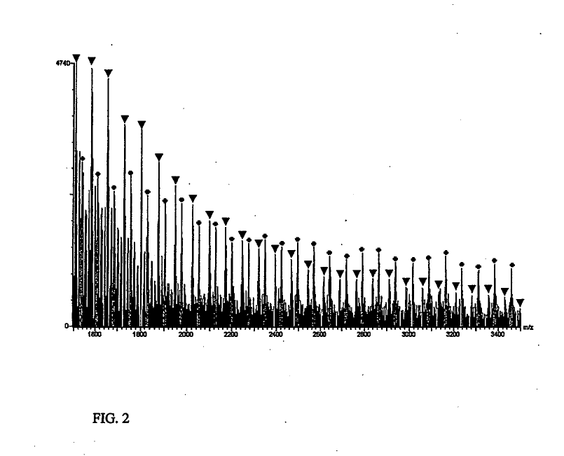

In order to determine if the same species are obtained if the gel is in an aqueous environment (as may occur if in contact with the skin for a prolonged period) the gel was extracted with water and the MALDI analysis repeated as above. The spectrum is shown in FIG. 2. The spectrum is very different from that shown in FIG. 1 and there are other chemical species present. This has been found to be due to the presence of small amounts of poly(ethylene glycol) (PEG) which may be present in the gel but is a comparatively minor component when the sample is extracted with chloroform. The PEG has been removed prior to quantitation of the silicone species. In the analysis of spectrum 2 a fit to the data is only possible if the cyclic species are a minor component compared to the linear siloxanes which, again, are methyl/hydroxyl and either methyl/methoxy- or methyl/methylol-terminated oligomers. The relative concentrations of these species may be estimated from the MALDI data and are shown in Table 1. This table contrasts the relative amounts of the cyclic and linear species removed by the two extraction media. It may be seen that, while the cyclic species are 60% of extractable oligomers by chloroform extraction, this drops to 40% when water is used for extraction.

Both the methyl/methylol or methyl/methoxy- and the methyl/hydroxy terminated linear species increase when water replaces chloroform for extraction. The ratio of linear to cyclic species is higher in the water extract than in the chloroform extract.

### Example 5

### Identification of Species Present on Gel Surfaces and Migrating Species

If the migration of the PDMS species from the gel to the skin is of importance in wound healing, then it is important to identify the species present both on the surfaces of the gels and which may migrate, not just those which may be extracted. The sensitivity of MALDI allows the small amount of material which is transferred by touching the gel to the target to be analysed by depositing a layer of matrix material and running the mass spectrum as described above.

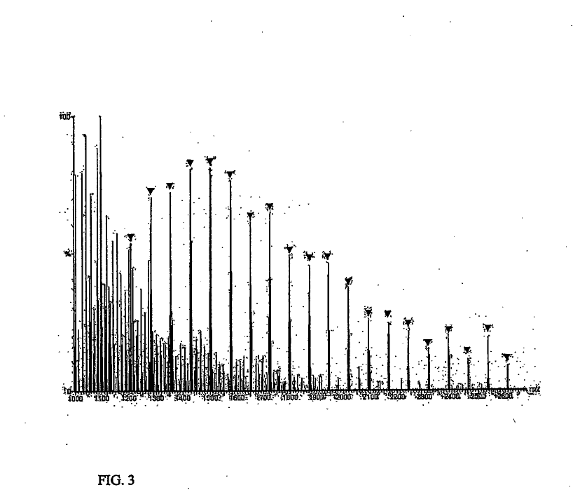

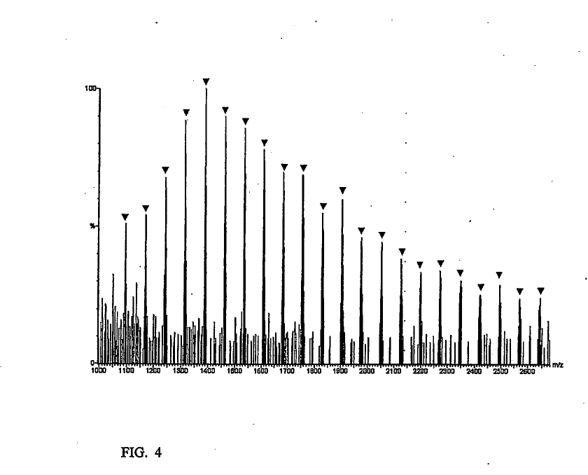

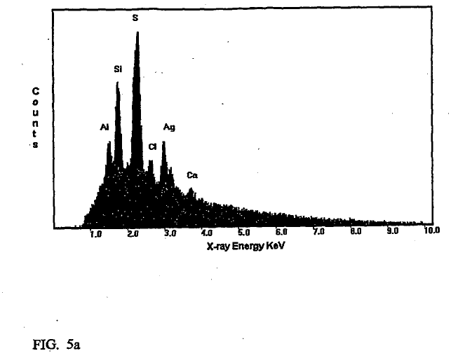

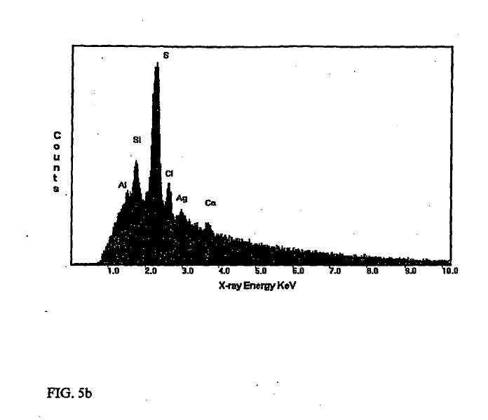

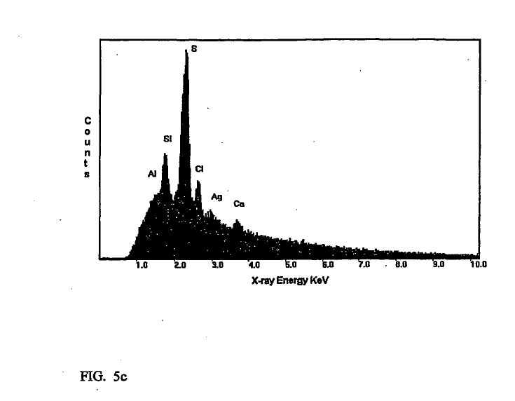

FIG. 3 shows the MALDI mass spectrum of the species transferred from a fresh sample of the Cica-Care®. As above the spectrum is again contaminated with PEG but the dominant silicone species are the cyclic oligomers. In the normal usage of the gel against the skin there will be water present and, in prolonged use, the skin will be frequently washed. In light of this the MALDI spectrum of the surface species after washing and touching the wet sample to the target was obtained. These results are shown in FIG. 4 and the dominant oligomer is the linear methyl/methylol or methoxy-terminated species.

The experiments involving direct contact between the gel and the MALDI target identify the low molecular weight species at the surface of the gel when either wet or dry, but do not show that they are able to migrate into skin or scar tissue. For this the inventors have used the model system of a gelatine matrix. This model system was chosen as gelatine provides a matrix of collagen and hypertrophic scarring results from over-production of collagen in the dermis. The gelatine provides a matrix of hydrolysed Type 1 bovine collagen which may be readily freeze-dried and sectioned in order to determine the distribution of silicon. While, in principle, since the MALDI spectrometer's instrumental software allows a line map to be constructed along a line of laser shots, MALDI may be used for mapping experiments, the low resolution of 100 μm limits MALDI spectrometer's usefulness in this application. To overcome the shortcomings of MALDI the inventors have used energy dispersive X-Ray analysis (EDX) in the Scanning Electron Microscope to provide an alternative semi-quantitative technique. The inventors carefully ensured the water in the gel was removed by freeze-drying, as required for EDX.

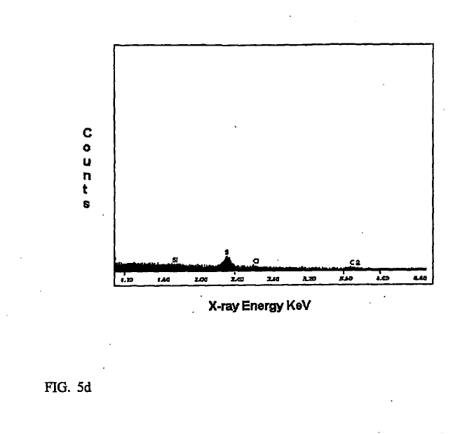

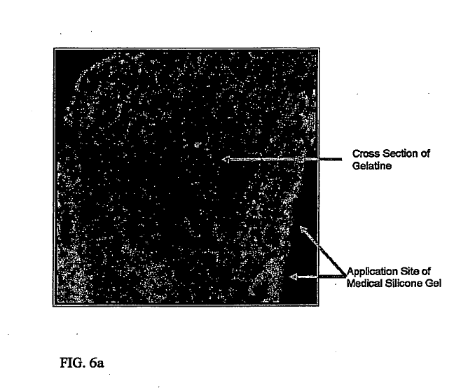

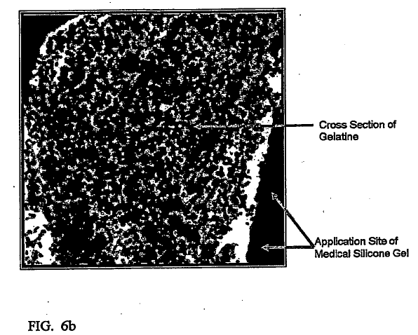

FIG. 5 shows representative EDX spectra from different positions, proceeding from the front surface (a), through the bulk (b) to the back surface (c) of a sectioned sample of gelatine after contact with a patch of silicone gel sheeting for 16 weeks. It is seen that the silicon signal is well resolved from the other bands. The sulphur and chlorine bands arise from the antibiotic added to the gelatine. A map may be constructed of silicon distribution over the entire thickness of the gel and this is shown in FIG. 6 at two levels of threshold sensitivity. (NB Silicon appears as light regions in these maps). The images show that there is migration of silicon into the collagen layer and there are some areas in the bulk where a high local concentration is achieved. It is noted from the map that the highest concentration occurs at the side in contact with gel, but a significant concentration is detected on the surface away from the gel as well as in the bulk of the gelatine. The front is the face to which the patch is applied, but the high concentration on the back surface suggests that there has been migration through the gelatine and aggregation on the back surface.

The siloxanes, being highly surface active, will aggregate at the air-gelatine interface, but there must be diffusion through either the water or the hydrophobic collagen in order to penetrate the gelatine. The cyclic and methyl-terminated linear species are highly hydrophobic and association with the collagen is expected. For example it has been reported that hydrophobic interaction between heme proteins and PDMS lead to denaturation (Anderson, et al., Biophysical Journal, 1995, 68, (5) 2091) and that small cyclics with n=4 (octamethyl cyclotetrasiloxane or D4) induce conformational changes in fibrinogenin and fibronectin which are wound healing proteins (Sun, et al., Biomaterials, 1998, 18, (24) 1593).

Based on these results, the presence of zones in the bulk with high concentration of silicone species would suggest strong hydrophobic interaction between the siloxane oligomers and the collagen in certain areas. However, an interesting result, noted earlier, is that while the surface of the silicone gel is rich in cyclic species, these are a minor component compared to the linear oligomers that are detected at the surface by MALDI when the gel is wet.

### Example 6

### Mechanism of Remediation of Hypertrophic Scarring by Mobile and Associative Species

It has been recently reported that linear silicones modified with hydrophilic groups will associate with proteins at interfaces and stabilize them against denaturation (Zelisko, et al., Proceedings-28th International Symposium on Controlled Release of Bioactive Materials and 4th Consumer & Diversified Products Conference, 2001, 2, 997). Whether simple hydroxy group termination is sufficient to achieve this stabilization or whether the silicone facilitates denaturation through inversion of the structure on hydrophobic interaction is of fundamental interest in a likely mechanism of remediation of hypertrophic scarring by these mobile and associative species. As there is only a limited amount of extrapolation which may be made from the hydrolysed collagen matrix to the dermal layer, studies have commenced on the species which may actually migrate into skin and scar tissue from a silicone gel sheet.

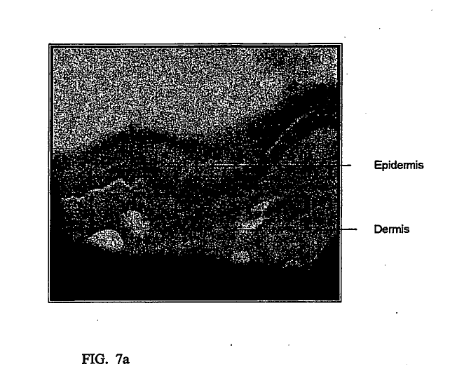

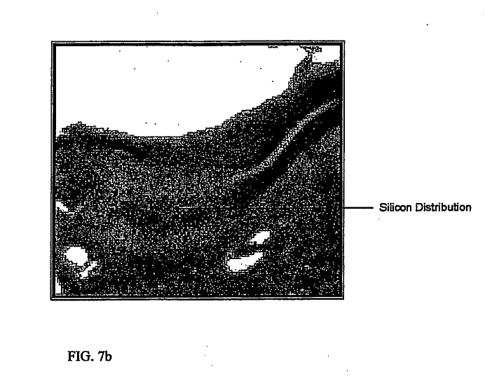

FIG. 7(a) shows a STEM photograph of a cross-section of scar tissue and the extent of the epidermis and the lower dermal layer. FIG. 7(b) shows the X-ray microanalysis map for silicon from the same cross-section. It is seen that silicon is widely distributed in the sample and that the highest concentration appears in the interfacial region between the dermis and epidermis. The concentration in the epidermis which has developed by re-epithelialisation following the burn is very low compared to the dermis and the distribution declines from the maximum at the interface towards the inner dermal layer.

It should be noted that silicon is present in healthy skin and is linked to collagen development which complicates the extension from the model system to skin. Additionally, both this silicon present in healthy skin and the silicon originating from the ubiquitous use of silicones in skin-care products will result in a high background against which measurements are to be made. The form of the silicon is not known and MALDI-MS analysis of the silicone species on skin is required.

### Example 7

### Ionisation and Analysis of the PDMS Species

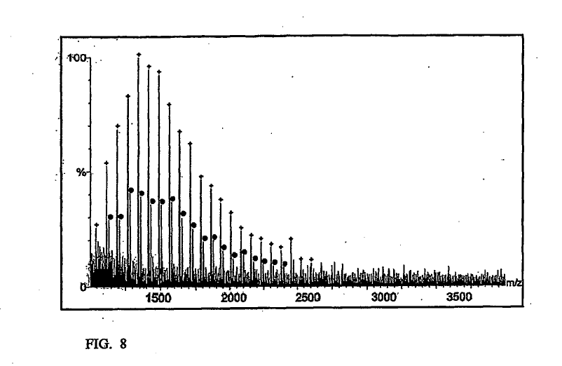

Studies carried out by the present inventors on scar tissue to which a chloroform extract has been applied, (which as noted in FIG. 3 consists of predominantly cyclic species) have shown that ionisation and analysis of the low molecular weight silicones may be achieved (but at poorer S/N). This is shown in FIG. 8, which is the MALDI mass spectrum of a sample from the surface of a burn scar to which the silicone had been applied. A fine spray of the matrix was applied to the partially dehydrated skin prior to analysis.

In another report of molecular imaging of biological samples using MALDI-TOF mass spectrometry, the direct analysis of tissue produced interference from signals from abundant molecules such as lipid and protein fragments around the lower molecular weight region (MW<1500) (Caprioli, et al., Anal. Chem., 1997, 69, 475). The present inventors have investigated, both skin with and without a matrix solution deposited onto it through analysis with MALDI-TOF mass spectrometry. No interfering biological species were detected, possibly because the analysis conditions had been optimised specifically for the PDMS oligomers. Thus in principle the inventors' studies of the model gelatine system may be directly extended to skin and scar tissue to determine if the cyclic or linear siloxane species are those which migrate under the conditions prevailing on the surface of a scar when a silicone gel patch is applied. Examination of FIG. 8 suggests that this may be determined by a careful comparison of the oligomers that are desorbed and analysed. Comparison of the MALDI spectra in FIG. 1 (the extract as applied) and FIG. 8 shows that the methyl/methylol-(or methoxy-) terminated oligomer is not desorbed from the scar tissue. There may be several explanations for this behaviour, but one possibility is that these oligomers have migrated preferentially into the epidermis and are strongly associated with the proteins or other extracellular matrix components.

The present inventors continued investigations of skin and scar tissue to determine more decisively the species which may migrate through the stratum corneum to the dermis and the effect this may have on the properties of the collagen. The challenge in direct MALDI analysis from sections of skin such as that shown in FIG. 7 is the limited spatial resolution of the Nitrogen laser pulse used for ionisation. This is typically 100 μm while the entire thickness of the epidermis after re-epithelialisation is much thinner than in healthy tissue and in FIG. 7 is ˜70 μm.

### Example 8

### Migration of Functionalised Silicones Through the Stratum Corneum

A full thickness skin extraction was delaminated to separate the dermis from the epidermis as per the method described in the prior art (Kligmann, et al., Arch. Dermatol., 1964, 88, 702). The delaminated stratum corneum epidermis was treated in a trypsin digest phosphate buffer solution to remove the viable tissue leaving the keratinized stratum corneum. The isolated stratum corneum was dried in a filter paper and freeze stored until required.

The average thickness of stratum corneum has been reported to be approximately 15-18 μm. The stratum corneum harvested from abdominal reduction had an average thickness of 20 μm as determined by electron microscopy. The structure of stratum corneum has been likened to a brick wall arrangement as well as to a more simplified diagonal channel structure. It can also be considered that the stratum corneum diffusion takes place through a lipid conduit of an actual length much greater that that of the stratum corneum thickness. Whatever description is used to describe the actual diffusion of molecules across stratum corneum, different researchers presently believe that hydrophobic substances can only permeate through the extracellular pathway.

The calculation of a diffusion coefficient for a permeant requires additional information other than the break across a membrane. The rate at which the penetrating species permeates at a steady state is also required. A two cell compartment is normally used in the determination of permeation rates, where the permeant is continuously being removed after it transverses the membrane. Attenuated total reflectance (ATR) analysis does not remove the penetrant as it emerges at the other end of the membrane, and can only measure the concentration of the permeant within a thin section or layer, in direct contact with internal reflection element (IRE). ATR can not measure the rate of permeation but it is useful in determining the diffusion coefficient. By definition the diffusion coefficient is a measure of the resistance by the stratum corneum to the permeation of a traversing substance.

Table 2 is a summary of the data obtained by the inventors from ATR analysis of stratum corneum treated with silicone medical gel and extracted low molecular weight silicone oil (left and right hand side of Table 2 respectively). The silicone medical gel experiment was performed at a different temperature than the extracted low molecular weight silicone oil (22° C. and 32° C. respectively). This demonstrated temperature dependency of the diffusion coefficient, verifying a Fickian diffusion profile.

The present inventors have shown that PDMS's diffusing from silicone medical gels are able not only to permeate the stratum corneum, but also to diffuse into the epidermis and dermis. However in hypertrophic scars the demarcation between the dermis and epidermis is not a clear one. Hypertrophic scars comprise highly disorganized collagen bundles; the stratum corneum is also considerably thinner than normal scar and healthy tissue. The cellular activity observed from thin section microscopy is not sufficient to predict any interaction between the silicone penetrant and fibroblast or monocytes. However, as silicones permeate in excess of 200 μm, it can be inferred that this hydrophobic material not only can, but will activate monocytes. It is known that silicone deactivates fibroblasts and that fibroblasts are responsible for both collagen and collagenase synthesis. Here it is believed that the only source of these higher levels of elemental silicon is due to migrating low molecular weight species of silicone oligomers from the applied silicone medical gel. This allows a pictorial visualisation of elemental silicon distribution from which one can infer the permeation of migrating silicone species. This correlation is essential in order to determine where within the hypertrophic scar tissue the low molecular weight silicone species accumulate.

In summary, while the stratum corneum is generally regarded as a non-permeable barrier, certain molecules are able to permeate across this barriers. The present inventors have identified a series of cyclic and linear polysiloxanes, in particular, substituted or ‘functionalized’ PDMS's which are able to migrate into the hydrophobic epidermis layers through to the lower hydrophilic dermis skin layers. It has been found that the nature of the substituent(s) or functionalised group(s) on the PDMS polymer are determinants in the rate and quantity of migration into the aqueous environment. Polysiloxanes with water soluble groups have been found to diffuse at a higher rate than hydrophobic polysiloxanes. Silicone polymers by nature are hydrophobic due to the high concentration of alkyl substituents on the siloxane backbone. However, even minor substitutions to any of these substituents can change the hydrophobic nature of the entire PDMS molecule. Hence by altering various substituents on the cyclic and linear PDMS polymers, a series of functionalized PDMS molecules may be tailored to allow passage through both the upper hydrophobic epidermis and lower hydrophilic tissues.

Without wishing to be bound by any particular theory, the present inventors contemplate that the mode of action of the functionalized PDMS's is by a causal cascade at both molecular and cellular levels. The migrating functionalized PDMS's permeate and diffuse across the stratum corneum and into healthy and scar tissue where the functionalized PDMS's interact with the extracellular matrix and proteinaceous component of the localized tissue. There are three key modes of action believed responsible for the efficacy in hypertrophic scar remediation.

### Monocyte Activation

It has been reported that human monocytes are activated in the presence of silicones, suggesting that the immune cell infiltrate increases macrophage activity. In post silicone medical gel treatment, the immune cell infiltrate from hypertrophic scar tissue shows a clear increase of macrophage activity (Borgognoni, et al., 2000,/www.medbc.com/annals/review/, 13, 164), suggesting that silicone medical gel acts as an activation mechanism for monocytes. It is known that plasma proteins when absorbed to silicone become denatured and activate monocytes (Naim et al., Colloids and Surfaces, 1998, 11, (½) 79). It is therefore contemplated that the aforementioned species may be used to induce fibroblast suppression.

Fibroblast Suppression

It has been suggested that silicone polymers deactivate and inhibit growth of human fibroblasts (McCauley, et al., J. Surg. Res., 1990, 49, 103). McCauley et al. have described human dermal fibroblast behaviour in the presence of silicone gel prosthesis polymers, primarily manufactured for breast implant applications. The polymer in question has the same configuration as the Cica-Care® silicone medical gel used by the Royal Brisbane Hospital's Burns Unit, for hypertrophic scar remediation. The findings by these group's work suggests that there appears to be a deactivation effect of the human dermal fibroblasts with reduced survivability as well as morphological change of the cellular component of the surviving fibroblasts. Although the mechanism of action is unknown, the significance of the role in scar remediation is appreciated. The present inventors therefore contemplate that the aforementioned species may be used to suppress fibroblast growth.

Collagenase Enhancement

Both monocytes and fibroblasts are responsible for regulation and synthesis of collagenase. It is known that, in the presence of excessive collagen deposition, both of the major collagenase regulatory pathways may be affected by active siloxanes and thus equilibrium restored. It is therefore contemplated that the aforementioned species may be used to induce collagenase enhancement.

### Example 9

### Synthesis of PDMS Intertissue Migratory Agents

Scheduled synthesis of the PDMS's shown in Tables 3-5 may be carried out using the synthesis of silicone polymers via ring opening polymerisation of a cyclic polysiloxane trimer or tetramer. Condensation polymerisation is an additional synthetic pathway for silicone polymers (Maravigna et al., Comprehensive Polymer Science, 1998, 5) whereby reactive hydroxyl groups are substituted at the terminus of the chains and subsequently undergo substitution reactions, thereby ‘functionalising’ the silicone polymer (Mark, Silicon-based Polymer Science: A Comprehensive Resource, 1990). The hydroxyl groups may be substituted for dimethyl and vinyl end groups. To obtain the gel property of these silicone sheets, PDMS may be cross-linked according to known methods (Ravve, Principles of Polymer Chemistry 1995). Cross-linking is accomplished at the vinyl end group sites through abstraction by free radicals which are generated by the decomposition of added peroxides (Hirshowitz, et al., Eur. J. Plast. Surg., 1993, 16, 5; Ravve, Principles of Polymer Chemistry 1995). The peroxide 2,4-dichlorobenzoyl may be used to cure silicone elastomers. Additionally, these polymers may be reinforced with an inner polymer mesh, such as poly(ethyleneterephthalate) or poly(tetrafluorethylene) (Ahn et al., Surgery, 1989, 106, 781 and Suare, et al., Dermatol. Surg., 1998, 24, 567).

In the ring opening polymerisation process, macrocyclic species are obtained in 10 to 15 wt % yields. Reactive functional end groups may be formed via reaction with water, alcohol, divinyltetramethyldisiloxane or tetramethyldisiloxane with chlorosiloxane end groups using the methods described in Colas (Colas, Chimie Nouvelle, 1990, 8, (30) 847). Other suitable reactions are described below;

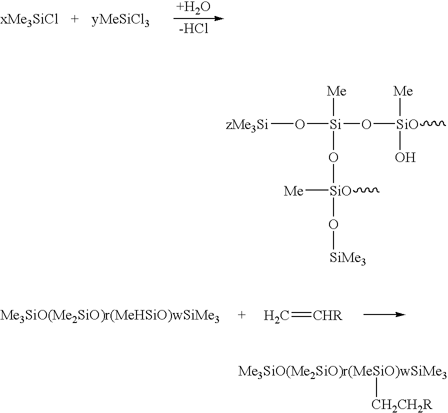

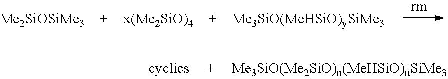

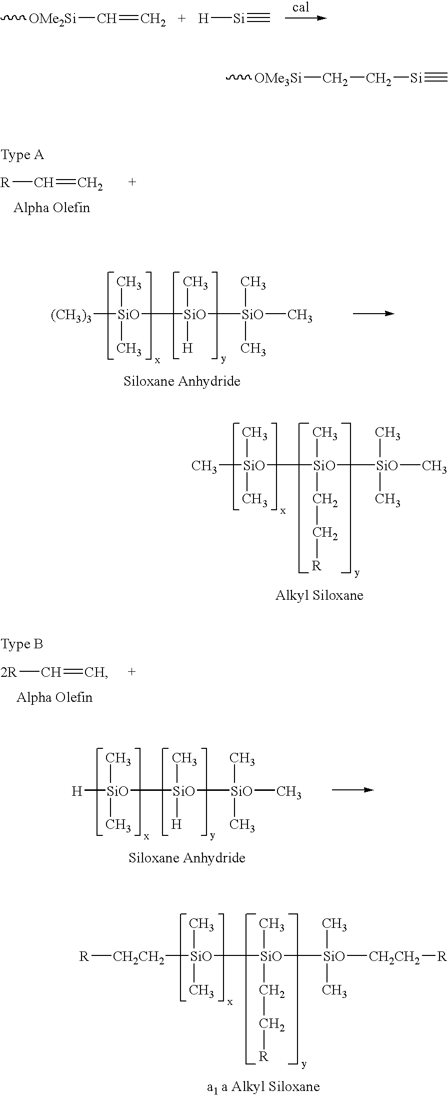

During the polymerization of PDMS, a series of oligomers of various molar masses are formed that result in a molecular weight distribution (MWD). Experimental measurements of molecular weight can only result in an average value and therefore several averages are expressed. The polydispersity (PD) of a polymer is the ratio of M_{w}/Mₙ it describes how close the masses of the oligomers that make up the polymer sample, are to each other. A narrow polydispersity ratio for example of ≈1.2 indicates that the distribution of the oligomers is close and the range of molecular masses is small. However a high PD, for example 3.0, indicates that the range of molecular masses is high and the disparity between the two ends of the mass spectrum is significant. For a normal distribution M_{w}/Mₙ≈2.

### Example 10

### Neo-Natal Foreskin and Hypertrophic Fibroblast in-Vitro Silicone Results: Cell Activity

Exposure to silicone fluids and silicone medical gels in-vitro is known to activate and deactivate monocyte and fibroblast cells accordingly (Kuhn et al. Int. J. Sur. Investig., 2001, 2(6), 443; McCauley, et al., J. Surg. Res., 1990, 49, 103; Naim et al., Colloids and Surfaces, 1998, 11, (½) 79). In the past the premise for this was that silicone fluids sat on the skin surface acting as a seclusion agent or perhaps affected the hydration of the site under treatment and did not penetrate into the viable tissue. However, as demonstrated by the inventors above, the silicones of the present invention permeate the stratum corneum. In addition to skin permeation, the inventors have also studied the interaction between functionalized silicone species and fibroblasts.

Seven species of low molecular weight silicones extracted from the Cica-Cares medical gel were chosen for their similarities in molecular weight range and functionalities to those extracted from medical gel patches, were tested in-vitro with two fibroblast primary cell lines. The silicones tested are identified as follows:
ethoxy end capped 3400 amu linear silicone with terminal functional groups; low viscosity; ethoxy endblocked dimethyl silicone; clear, colourless to slightly hazy liquid; Wt./Gallon=8.0 lbs.; viscosity at 25° C.=68 cSt.; flash point (P.M.C.C)>200° F.; 100% Active; 2.8% (weight) ethoxy content; (referred to herein as ethoxy);
methoxy end capped 3500 amu, linear silicone terminal functional groups (referred to herein as methoxy),
GP582: hydroxyl end capped 18000 amu linear silicone terminal functional group; nominal viscosity of 750 cSt at 25° C.; clear, colourless liquid; Wt./Gallon=8.1 lbs.; specific gravity=0.98; 100% silicone;
GP426: hydroxyl end capped 3400 amu; linear silicone terminal functional groups; nominal viscosity of 100 cSt at 25° C.; Wt./Gallon=8.1 lbs.; specific gravity=0.99; 100% silicone;
GP507: branched carbinol 3000 amu; branched functional group; 100% active carbinol functional silicone polymer; clear, colourless to straw liquid; wt./gallon=8.0 lbs; viscosity at 25° C.=293 cST; flash point (P.M.C.C)>200° F.; specific gravity=0.98; OH content (carbinol)=3.55%;
GP226: pendant rake structure polyol 4300 amu branched functional group; water-dispersible; resistant to breakdown by hydrolysis due to the presence of stable SI—C bonds between silicone and polyol block sections; low surface tension; water dispersible; non-hydrolyzable; low freeze point; 100% active polyoxyethylene (EO) modified dimethylpolysiloxane block copolymer; clear to hazy, colourless to straw liquid; wt./gallon=8.0 lbs; viscosity at 25° C.=90 cST.; flash point (P.M.C.C)>300° F.; specific gravity=1.04; 100% active; 42% silicone; freezing point 32° F.; and
GP218: pendant rake structure polyol 5100 amu, branched functional group; dimethyl/silicone polyol copolymer;

It is appreciated that values of m, n and y in formula (I) may be selected so that the average molecular weight of compounds defined by formula (I) match or closely match the molecular weights listed for the compounds tested as listed above.

The silicones tested are all available from Genesee Polymers Corporation, G-5251 Fenton Road, Flint, Mich., 48507, United States of America.

The main characteristics of the silicones tested are as follows:
a) four of the silicone compounds are terminally functionalized and partially miscible in aqueous environments (the ethoxy, the methoxy, GP582, and GP426)
b) the only silicone fully miscible/dispersible in an aqueous environment is an ester branched low molecular weight silicone with a polyethylene glycol (PEG) functional group (sample GP226).

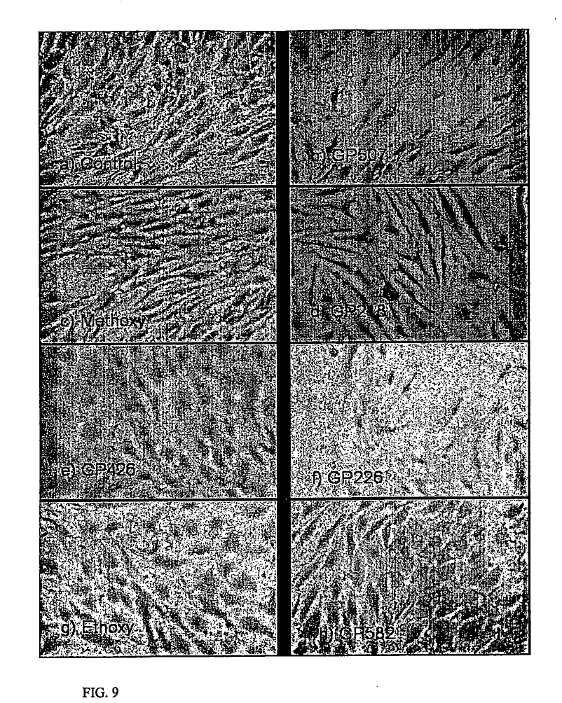

A control group was also incubated under the same conditions as the treated fibroblast cells. FIG. 9 contains 8 panels labelled a)-h) which show the results of the foreskin fibroblast primary cells stained with Sulphorhodamine B.

The fibroblast cells were derived from infant foreskins and expanded as required. The cells were seeded in Dulbecco's modified Eagles medium 10% foetal calf serum and streptomycin as a fungicide plus Gibcobrl (Penicillin and Streptomycin) Cat# 15140-122 lot# 1007346. Tissue culture plastic 96 well plates were seeded with 2×10³ cells per well and allowed to rest for 48 hours (four replicates), the plates were separated into distinct treatment arms for treatment with one of the 7 silicones listed above. The cells were inoculated with the respective silicones after the period of rest and allowed to incubate until the control arm became confluent.

Once confluence was achieved in the control arm the cells were then fixed and stained with Sulphorhodamine B as per method described by Raman et al. (Raman et al.), and a representative of each treatment arm was photographed. The cells were then solubilized as per the method described by Raman et al (Raman et al., ibid). The solubilized samples were placed in a microplate reader and analysed at a wavelength of 540 nm.

Solubilization of the samples was achieved by modifying methods from Kiernan et al, Kiernan, J. A. and Lowe et al (Kiernan et al., 2001, Biotech. Histochem., 76(5-6), 261; Kiernan, J. A., available at http://www.histosearch.com/histonet/OctOl/Re.siriusredforcollagenra.html; and Lowe et al., 1997). In short, a caustic solution 50/50 of 2.5M NaOH and methanol was used and the plates were then placed in a microplate reader at 540 nm

The results depicted in FIG. 9, with the non-specific protein dye sulphorhodamine B, show the low molecular weight functionalized silicones affect natural development of fibroblast cells. The effect of these siloxanes is apparent on visual inspection of FIG. 9., wherein the fibroblast cells shown in panels b), d), and f), those treated with GP 507, GP 218 and GP226 respectively, show a discrete change in cellular morphology compared to the control group shown in panel a).

The cells stained in the experiment depicted in FIG. 9 were also analysed for absorbance at 540 nm using a BioRad Microplate reader Benchmark Plus spectrophotometer with a Xenon light. The results of the spectrophotometry analysis are shown in Table 6.

The data shown in Table 6 shows that the control and GP507 groups are almost identical in magnitude. However, these are results from experiments using the non-specific protein dye sulphorhodamine B and are indicative of total protein production by a cell and is not indicative of specific protein amount, such as collagen amount.

### Example 11

### Neo-Natal Foreskin and Hypertrophic Fibroblast In-Vitro Silicone Results: Collagen Production

To further investigate the influence of the siloxanes of the invention on protein production, primary cells from the same passage and batch incubated under the same conditions, detailed in Example 10 above, were inoculated with the seven functionalized silicones listed above. Hypertrophic derived fibroblast cell primary cell lines obtained from donors suffering from hypertrophic scars on the forearm were also analysed.

The cells were treated as above with the exception that each replicate was the result of a passage and that the cells were stained with the collagen specific stain Sirius red.

As discussed above, the results in FIG. 9 and Table 6 with the non-specific protein dye sulphorhodamine B reveals information on total protein synthesis, and not on the type of protein synthesised. In order to determine the collagen activity of the fibroblast cells treated with low molecular weight functionalized silicones the inventors formulated a new protocol.

While collagen is regularly stained for in histological tissue analysis, conventional histology protocols were not suited to the requirements of the present investigation. In order to selectively determine the collagen production capabilities of primary fibroblast cells in-vitro the inventors utilized the collagen specific stain Sirius red (chemical index name and number: Direct red 80, 35780) which due to its ease of application is commonly used in histological preparations (Kiernan et al. Biotech. Histochem., 2001, 76(5-6), 261).

The method pioneered by the inventors in order to stain the fixed primary fibroblast cells is based on an adaptation of three histological prep-methods. In the inventors method of staining, sirius red staining solution was prepared by dissolving 0.5 g of Sirius red F3B, (direct red 80 C.I. number 35780; empirical formula C₄₅H₂₆NO₁₀O₂₁S₆Na₆ formula weight 1373.125 amu), in 500 mL of saturated aqueous picric acid. A rinse solution was prepared by mixing 5 mL of glacial acetic acid in 11 mL of distilled water. The cells were fixed by the addition of 25 μL of cold 50% trichloroacetic acid (TCA, 4° C.) on top of the growth medium and incubated for 1 hour at 4° C. The cells were then gently rinsed with water and then each well was inoculated with 50 μL of Sirius red solution and incubated at room temperature for 30 minutes. The wells were drained and lightly rinsed with distilled water and representative wells were then digitally photographed.

Solubilization of the samples was achieved by modifying methods from Kiernan et al., Kiernan, J. A., and Lowe et al (Kieman et al., 2001, Biotech. Histochem., 76(5-6), 261; Kiernan, J. A., available at http://www.histosearch.com/histonet/OctOl/Re.siriusredforcollagenra.html; and Lowe et al., 1997). In brief, a caustic solution of 50/50 2.5M NaOH and methanol was used and the plates were then placed in a microplate reader at 540 nm

As mentioned above, two primary cell lines were utilized to examine the influence of the functionalized silicones on collagen production. Firstly, foreskin primary fibroblast cells (FF), similar to as used above, were used. Secondly, hypertrophic derived primary fibroblast cells (HF) were used. Two cell lines were used in order to determine if there is any significant adverse reaction by either cell type (FF or HF) to the low molecular weight silicones.

This line of investigation utilized a different protocol to inoculate the primary cell lines. Instead of inoculating all replicates at the same time, each replicate was inoculated following a passage of the primary cells. This procedure ensured that a new generation of cells was tested each time following the split of confluent cells.

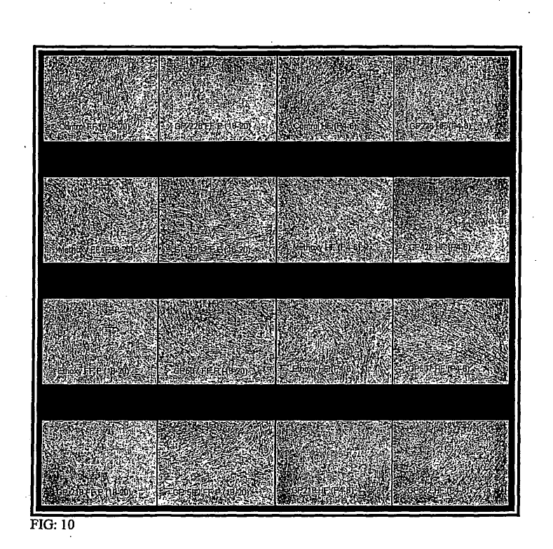

In this experiment three replicates were performed. The first replicate foreskin fibroblast primary cells (HFF1) used in this experiment was passage 18 (P 8), whilst the first replicate of hypertrophic derived primary cells (HSF1) was passage 4 (P4). Tables 7-12 and FIG. 10 depict the results, including the micro-plate read outs, for these experiments. HFF2 and HFF3 are the second and third passages respectively (i.e. passage 19 and 20 respectively) of the foreskin fibroblast primary cells. Similarly, HSF2 and HSF3 are the second and third passages respectively (i.e. passage 5 and 6 respectively) of the hypertrophic derived primary cells.

The cells were treated as above with the exception that each replicate was the result of a passage and that the cells were stained with Sirius red, which is a collagen specific stain.

Levels of collagen in solution can be determined by staining with sirius red and analysing the absorbance at 540 nm. To measure absorbance, the acidic stain was first solublized in NaOH-Methanol (2.5M) mixture before analysis in the microplate reader at 540 nm. The averaged results of the absorbance studies are tabulated in Table 13. The fixed and stained plates of each individual experiment were re-solubilized and analysed on a spectrophotometer for absorbance at 540 nm as described above. FIG. 10 shows representative images of the fixed and stained primary cells with sirius red.

The results of the in-vitro experiment, as shown in FIG. 10 and Tables 7-13, show that each silicone tested brought about a down regulation of fibroblasts in the cells treated and in this regard concur with the results obtained by Kuhn et al. and McCauley et al. (Kuhn et al. Int. J. Sur. Investig., 2001, 2(6), 443; McCauley, et al., J. Surg. Res., 1990, 49, 103). All silicones species examined brought about a down regulation with the most dramatic effect being caused by GP226.

On visual examination of FIG. 10 alone it can be concluded that the two most effective functionalized silicones are GP218 and GP226. Both of these compounds are silicone-polyol functionalised polymers. The miscibility or water dispersion capability of these polymers is related to their molecular weight, the larger the molecular weight of the polymer the less dispersible or soluble the polymer becomes.

While not wanting to be bound by any one theory, one explanation for the effectiveness of GP218 and GP226 is that since they are easily dispersible in aqueous solutions they form an envelope around cells, preventing the cells from nutrient intake.

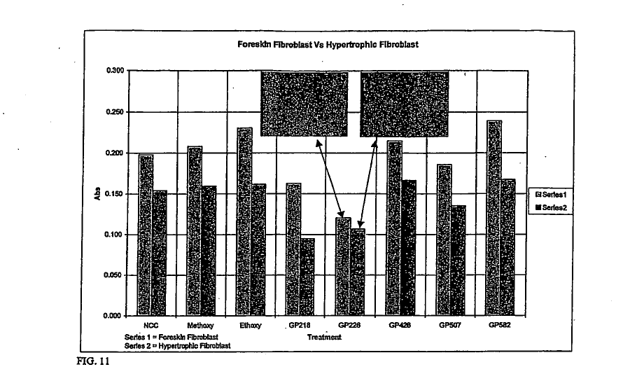

The highlighted rows in Table 13 and the corresponding columns in the bar graph FIG. 11, show that the average collagen production levels by the fibroblast cells treated with the GP218, GP226 and GP507 functionalized silicones are lower compared to the average control. Of considerable interest is the result of the carbinol functionalized silicone, GP507. This product appears to down regulate collagen production without inhibiting cell proliferation which could be of significance for early treatment of the wound healing process, particularly for burn injuries.

Dr Fiona Woods from the burns unit at WA Hospital has reported that early silicone treatment intervention as soon as re-epithelization occurs prevents hypertrophic development and excessive wound contracture. However this verbal communication requires verification by a medical trial.

Given that in the early history of silicone medical gel treatment there were reports of these patches being applied before re-epithelization caused severe wound infection and exudate accumulation, a cream or ointment would be better suited to begin treatment as early as pre-epithelization in order to prevent excessive scar development and wound contracture. It is desired that fibroblast cells be allowed to proliferate and that collagen production be limited to the constraint of natural collagen turnover in order to allow a normal wound maturation process.

### Example 12

### Cream Formulation Incorporating Functionalized Silicones

The functionalized silicones of the invention are suitable for inclusion in a composition for topical administration. GP218, GP226 and GP507 have been incorporated into a cream formulation. The formulations are based on an aqueous emulsion where the functionalized silicone is the active ingredient and is dispersed to a minimum of 30% content of the composition's overall weight (percent by mass).

A particularly suitable cream formulation for topical administration, that has been prepared by the present inventors containing GP218, GP226 and GP507 respectively, as the silicone according to formula (I) above, comprises:

| | |
|---|---|
| medical grade virgin olive oil | 12.0000% |
| glycerine | 8.0000% |
| silicone according to formula (I) above | 30.0000% |
| refined lanolin | 6.0000% |
| citric acid | 0.8000% |
| urea | 0.8000% |
| vitamin A | 0.0005% |
| cithrol GMS (glyceryl stearate) | 12.0000% |
| polawax GP200 (cetearyl alcohol, PEG 20 stearate) | 8.0000% |
| solvent (e.g. ultra pure water (Millipore)) | 22.3995% |

This is a basic formulation which can be combined with other known components. A person of skill in the art understands appropriate percentage amounts in which the components can be varied.

### Example 13

### Clinical Studies in Porcine Model

The inventors have applied to test the functionalized silicones of the invention, particularly those shown above to affect collagen production, GP218, GP226 and GP507, in a clinical study. The protocol for the clinical study is outlined below.

A porcine model is conventionally used in clinical studies of burns. Nine large white pigs are to be used in the first year of the study. Pigs are to be used as it is widely believed that the pig has skin most similar to that of the human (Meyer et al. Curr Probl Dermatol. 19781; 7:39-52) and that the pig represents the best animal model for human wound healing studies (Sullivan et al Wound Repair Regen. 2001 9:66-76).

Nine animals have been chosen to give three animals in each group (group 1: treatment with Cica-Care® gel, group 2: treatment with LMW Silicones of the invention; group 3: treatment with LMW silicones underneath the Cica-Care® gel). Three animals is the minimum number needed for statistical analysis. Two wounds are to be present on each animal, allowing for six wounds in total for analysis in each group.

### Experiment 13-1

This experiment will test the efficacy of a low molecular weight (LMW) silicone in reducing scarring after a deep dermal partial thickness burn.

All experimental pigs will be delivered to the animal house at least 5 days before the beginning of the experiment. All pigs will be administered 1 mg/10 kg of Stresnil™ prior to transport to reduce stress of new environment and mixing with potentially unknown pigs. On arrival they will be introduced to a moistened standard pellet diet. Animals will be held in individual enclosures of 2 square meters each to prevent them from chewing each others dressings and wounds.

The enclosures allow the experimental animals to see each other, minimizing isolation. The environment will be altered to allow environmental enrichment, such as large rubber balls, bowling balls, tyres etc, for the pigs to play. If amenable, the pigs will be allowed to run in the dog enclosure outside.

All pigs will be anaesthetised using a mixture of 13 mg/kg Ketamine and 1 mg/kg Xylazil (intramuscular) for induction followed by insertion of size 3 or 4 laryngeal mask airway (LMA) and ventilation with a halothane/oxygen mixture.

Injections are to be given to the pigs safely while minimizing stress to the animals. If not already, the staff responsible are to be trained in this regard. The hair on the upper back of the experimental animals will be clipped and the skin rinsed with clean water prior to wounding. All procedures will be performed using sterile technique in an appropriate research centre.

Buprenorphine (0.01 mg/kg) will be administered (intra muscular) at the beginning of the surgery. Experimental animals will be reassessed on the night of surgery (approx. 4-8 hours after the Buprenorphine dose) and if in obvious distress will be administered another dose. If the animal is sleeping they will not be disturbed. Buprenorphine will be administered on the following day and after if required. Behavioural assessment will be used to determine if analgesia is required (i.e. alertness, interest in surroundings and food etc).

Wounds will be created according to our method of creating deep dermal partial thickness burns as approved previously (#P&CH 728/03 and Modification 19/2/04). A hot water scalding device, comprising a Schott Duran bottle with the glass bottom removed and replaced with cling wrap is to be used. The bottle is filled with water and heated to 92° C. in a microwave. The bottom of the cling wrap is then to be placed in contact with the pig skin for 15 sec. Two burns will be created on each animal, in the dorsal/flank area.

After the burns are created the animals will be dressed with Jelonet and Melolin with the dressings changed weekly until re-epithelialisation (5-6 weeks). After this re-epithelialisation point, the treatments will begin. There will be 3 treatment groups with 3 animals in each. The same treatment will be used on each animal in the one group.

Group 1: Burn with Cica-Care® as Treatment

These burns (3 animals) will be covered with Cica-Care®, a dressing used in the hospital burns units for scar management. The dressings will be changed once a week. This treatment group will show the effect of the present treatment used in burns units today on burn scars.

Group 2: Burn with LMW Silicone as Treatment

These burns (3 animals) will be treated with a LMW silicone cream daily. The LMW silicone cream could be a cream as described in Example 12 above. The wounds will be examined once a week. This treatment group will show the effect of the inventors' new LMW silicone cream on burn scars.

Group 3: Burn LMW Silicone Underneath Cica-Care® as Treatment

These burns (3 animals) will be treated with a LMW silicone cream/Cica-Care® gel combination therapy. The LMW silicone will be applied to the wound daily, with Cica-Care® gel placed over the top. The wounds will be examined once a week. This treatment arm will show if there is any benefit of a combination therapy.

At weekly intervals after the thermal injury, the dressings will be removed from all three groups and the wounds photographed. At this time when the pigs are sedated, blood may also be taken for analysis of inflammatory markers. This procedure will occur weekly with the scar maturation monitored by two different observers. The photographs and clinical notes taken at each dressing change will be used to compare the healing in each wound. Clinical markers, such as scar colour (vascularity), scar profile (amount scar is raised above normal skin surface), amount of hair, size of wound, and amount of infection will be recorded as part of our clinical assessment scale.

The experimental animals will be kept for three months after the treatment regime begins. At this point (3 months+6 weeks) the pigs will be euthanized and tissue collected. Burned and control unburnt tissue will be collected and fixed in 10% buffered formalin and blocked in paraffin. Sections of 4 μm thickness will be stained with haematoxylin and eosin and examined in a blinded manner by an experienced histopathologist. The tissue will be scored for extent of histopathological damage, using markers such as the number of fibroblasts, alteration of interstitial tissue, epidermal thickness, number of hair follicles and alteration in papillary and reticular dermis. The processed tissue may also be used in the future for immunohistochemical analysis. Extra burned and normal tissue will be collected to perform tensile skin strength analysis. We will also collect burned and control tissue and freeze in liquid nitrogen or on dry ice for RNA, DNA and protein analysis.

For all weekly dressing changes and in circumstances where the pigs have removed their dressings we will sedate the animals while new dressings are applied. A 5.2 mg/kg ketamine/0.4 mg/kg xylasine dose (40% of the dose used to induce anaesthesia) will be used (#P&CH 728/03 Modification 15/4/04). This decreases the amount of stress the animals suffer while they are being held still and also reduces the risk of injury to staff when the pigs try to escape being held. The sedation provides adequate analgesia in circumstances when dressings being changed become stuck to the wound or it is necessary to remove the scab over the wound to see the healing underneath.

All staff conducting the experiments are to have learned pig handling techniques and to be confident and experienced with various techniques used to hold (e.g. with snares), carry, inject and bleed pigs.

Pain Management

The thermal injury will cause some distress. To eliminate this pain a combination anaesthetic technique, as discussed above, is used. Ketamine not only provides dissociative anaesthesia, but is also a very useful analgesic, and is used frequently in the military arena. Buprenorphine, a long-acting analgesic has been used for immediate post-operative analgesia and has proved successful for this type of injury. The animals are to be monitored daily (usually at least a couple of times a day) and buprenorphine can be administered if the animals are in discomfort (not envisaged to be necessary regularly). Constant assessment of the animals is performed.

The animal technicians are to be experienced and are to provide expert care to all animals. A distress chart for the pigs is to be utilized which has been developed previously by the inventors for a lamb burn model.

A 5% body surface area burn in a child is a significant injury, and comparable to the burns to be administered to the experimental animals. Such a burn will cause long term sequelae. However, it is small enough that these children would not require hospitalisation, and simple analgesics such as paracetamol (for a human) would be adequate to control any discomfort. No intravenous fluids would be used in this size burn in a child or adult, and resuscitation would not be required. Thus the size of injury is sufficient to investigate and trial new therapies, but not large enough to cause serious discomfort.

Feeding of Experimental Animals

A conventional feeding protocol is to be used, such as the one used at the Herston Medical Research Centre, and the feed to be obtained from the same source as it is currently obtained. The environment will be altered to allow environmental enrichment, using items such as large rubber balls, empty milk containers, tyres, dog toys etc, in which the pigs can play. If they are amenable, the pigs will be allowed to run in the outside dog enclosure before their wounds are created and after their wounds have healed.

Euthanasia and Disposal

At the end of the experiment the pigs will be euthanized with an overdose of Lethobarb (sodium pentabarbitone) (½ ml/Kg IV). All animals will be frozen until collected and incinerated.

Alternative Techniques

There is a large amount of in vitro data concerning potential beneficial compounds to improve treatment for burn injury. However, an animal model is necessary to develop techniques required and support the in vitro data. The current model is a superior large animal burn model and is the most appropriate to test first aid treatments on. Because the animal burn model has a reproducible, consistent injury, healing agents can be compared against each other effectively. After testing has occurred in our animal model, this product could proceed to human clinical trials, if proved effective in reducing scar.

Compliance with Current Queensland Animals Care and Protection Act and the Current NHMRC Australian Code of Practice for the Care and Use of Animals for Scientific Purposes

The protocol in this example complies with the current Queensland Animals Care and Protection Act and the current NHMRC Australian Code of Practice for the Care and Use of Animals for Scientific Purposes.

### General

Once synthesized, the functionalised PDMS intertissue migratory agents may be characterised and tested using the methodology of Examples 1 to 13 as herein described.

The active intertissue migratory agents of formula (I) and a composition comprising the agent are to be present in an amount sufficient to remediate scar tissue. Suitable dosages of the intertissue migratory agents of formula (I) and the pharmaceutical compositions containing such agents may be readily determined by those skilled in the art.

It will be appreciated by the skilled person that the present invention is not limited to the embodiments described in detail herein, and that a variety of other embodiments may be contemplated which are nevertheless consistent with the broad spirit and scope of the invention.

The silicones of the present invention are considered non-hazardous. The silicones of the invention are useful in the control of scarring and are of major functional, psychological and aesthetic significance to all physical trauma survivors. In particular, the ability of the silicones of the invention to limit hypertrophic scar formation means survivors of burns injuries can go on to live a normal life. Additionally, the silicones of the invention may be more effective and less expensive than prior art therapies.

TABLE 1

| | CHCl₃ % Extracted | Water % Extracted |
|---|---|---|
| Total Amount of Silicone | 36.00 | 4.00 |
| Extracted From Gel | | |
| Cyclic PDMS | 60.00 | 40.00 |
| —CH₃ Terminated PDMS | 26.00 | 4.00 |
| —OH or —OCH₃ Terminated | 14.00 | 56.00 |
| PDMS | | |

TABLE 2

| | |
|---|---|
| D-ATR @ 22 C. & | ZnSn ATR @ 32 C. & |
| 40% RH | 60% RH |

| Time | Peak | Time | Peak |
|---|---|---|---|
| Hrs | Height | Hrs | Height |
| 0.00 | 0.00 | 0.00 | 0.00 |
| 47.17 | 0.00 | 0.20 | 0.00 |
| 91.83 | 0.00 | 16.00 | 0.00 |
| 123.07 | 0.00 | 19.00 | 0.00 |
| 240.87 | 0.00 | 41.00 | 1.29 |
| 265.22 | 5.50 | 46.00 | 1.74 |
| 312.55 | 10.00 | 66.00 | 4.12 |
| 386.81 | 12.50 | 72.00 | 4.35 |
| 409.42 | 32.50 | 88.00 | 5.44 |
| 456.97 | 37.50 | 94.00 | 5.59 |
| — | — | 110.00 | 7.14 |
| — | — | 140.00 | 7.52 |
| — | — | 166.00 | 8.26 |
| — | — | 186.00 | 9.00 |
| — | — | 191.00 | 6.26 |
| — | — | 202.00 | 7.34 |
| — | — | 228.00 | 8.67 |
| — | — | 234.00 | 8.41 |
| — | — | 252.00 | 9.29 |
| — | — | 255.00 | 9.76 |
| — | — | 299.00 | 18.27 |
| — | — | 323.00 | 22.75 |
| — | — | 329.00 | 23.63 |
| — | — | 349.00 | 41.19 |
| — | — | 372.00 | 49.32 |
| — | — | 399.00 | 51.83 |
| — | — | 497.00 | 51.83 |
| — | — | 540.00 | 51.14 |

TABLE 3

| R | R′ | R″ |
|---|---|---|
| —CH₃ | —CH₃ | —CH₃ |
| OU | OU | OU |
| —CH₂OH | —CH₂OH | —CH₂OH |
| —OCH₂CH₃ | —OCH₂CH₃ | —OCH₂CH₃ |
| —OCH₃ | —OCH₃ | —OCH₃ |
| —OH | —OH | —OH |
| -UₙOH | -UₙOH | -UₙOH |
| -UₙOUₙ′ n = 1-7 | -UₙOUₙ′ n = 1-7 | -UₙOUₙ′ n = 1-7 |
| see PMDS-PEG | see PMDS-PEG | see PMDS-PEG |
| example below | example below | example below |

Wherein U and U′ = CₙH₂ₙ₊₁, CₙH₂ₙ₋₁ or CₙH₂ₙ₋₃
n = 20-60
m = 6
l = 7

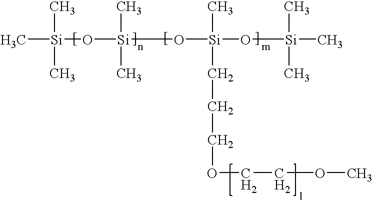

TABLE 4

| R | R′ | R″ |
|---|---|---|
| U = U, U′ and U″ = | U = U, U′ and U″ = | U = U, U′ and U″ = |
| CₙH₂ₙ₊₁, CₙH₂ₙ₋₁ or | CₙH₂ₙ₊₁, CₙH₂ₙ₋₁ or | CₙH₂ₙ₊₁, CₙH₂ₙ₋₁ or |
| CₙH₂ₙ₋₃ | CₙH₂ₙ₋₃ | CₙH₂ₙ₋₃ |
| —UCOOU′ | —UCOOU′ | —UCOOU′ |
| —COOU | —COOU | —COOU |
| —UCOOCOU′ | —UCOOCOU′ | —UCOOCOU′ |
| —COOH | —COOH | —COOH |
| —UCOOH | —UCOOH | —UCOOH |
| —UCOX | —UCOX | —UCOX |
| (X = Cl F Br I) | (X = Cl F Br I) | (X = Cl F Br I) |
| —COX | —COX | —COX |
| (X = Cl F Br I) | (X = Cl F Br I) | (X = Cl F Br I) |
| —UCO₂R′ | —UCO₂R′ | —UCO₂R′ |
| —COOCOU | —COOCOU | —COOCOU |
| -ArylU | -ArylU | -ArylU |
| -Aryl | -Aryl | -Aryl |
| -ArylUU′ | -ArylUU′ | -ArylUU′ |
| -ArylUU′U″ | -ArylUU′U″ | -ArylUU′U″ |

TABLE 5

| R | R′ | R″ |
|---|---|---|
| —NH₂ | —NH₂ | —NH₂ |
| —UNH₂ | —UNH₂ | —UNH₂ |
| —NHU | —NHU | —NHU |
| —NUU′ | —NUU′ | —NUU′ |
| —NO₂ | —NO₂ | —NO₂ |
| —UNO₂ | —UNO₂ | —UNO₂ |
| —UCONH₂ | —UCONH₂ | —UCONH₂ |
| —CONH₂ | —CONH₂ | —CONH₂ |
| —UCONHU′ | —UCONHU′ | —UCONHU′ |
| —CONHU | —CONHU | —CONHU |
| —UCONU′U″ | —UCONU′U″ | —UCONU′U″ |
| —CONU′U″ | —CONU′U″ | —CONU′U″ |
| —F | —F | —F |
| —Cl | —Cl | —Cl |
| —Br | —Br | —Br |
| —I | —I | —I |
| —PO₄H₃—PO₄H₃₋ₙ | —PO₄H₃—PO₄H₃₋ₙ | —PO₄H₃—PO₄H₃₋ₙ |
| —PU₃—PU′U″U″′ | —PU₃—PU′U″U″′ | —PU₃—PU′U″U″′ |
| —PO₄H₃₋ₙnU | —PO₄H₃₋ₙnU | —PO₄H₃₋ₙnU |
| —SH | —SH | —SH |
| —SO₂ | —SO₂ | —SO₂ |
| —SO₃H | —SO₃H | —SO₃H |

Wherein U, U′, U″ and U″′ = CₙH₂ₙ₊₁, CₙH₂ₙ₋₁ or CₙH₂ₙ₋₃

TABLE 6

| Plate A | | Plate B | | Plate C | | Plate D | | Average | |
|---|---|---|---|---|---|---|---|---|---|
| GP507 | 26192 | GP507 | 33162 | GP507 | 40264 | GP507 | 60926 | GP507 | 43622 |
| Methoxy | 23431 | Methoxy | 22875 | Methoxy | 23235 | Methoxy | 44774 | Methoxy | 30387 |
| GP218 | 42684 | GP218 | 28498 | GP218 | 47185 | GP218 | 45073 | GP218 | 42616 |
| GP426 | 49672 | GP426 | 19724 | GP426 | 25666 | GP426 | 40771 | GP426 | 23675 |
| GP226 | 4672 | GP226 | 5922 | GP226 | 5592 | GP226 | 10523 | GP226 | 57137 |
| Ethoxy | 59408 | Ethoxy | 23915 | Ethoxy | 30197 | Ethoxy | 35409 | Ethoxy | 35756 |
| GP582 | 55987 | GP582 | 19571 | GP582 | 21876 | GP582 | 33066 | GP582 | 30907 |
| Control | 41865 | Control | 44471 | Control | 42779 | Control | 45402 | Control | 43783 |

TABLE 7

| HFF1 | 1 | 2 | 3 | 4 | 5 | 6 | |
|---|---|---|---|---|---|---|---|
| NCC | 0.141 | 0.156 | 0.159 | 0.189 | 0.234 | 0.140 | |
| Methoxy | 0.149 | 0.216 | 0.165 | 0.179 | 0.259 | 0.173 | 0.190 |
| Ethoxy | 0.151 | 0.151 | 0.187 | 0.217 | 0.273 | 0.289 | 0.211 |
| GP218 | 0.153 | 0.403 | 0.210 | 0.133 | 0.339 | 0.137 | 0.229 |
| GP226 | 0.092 | 0.106 | 0.148 | 0.108 | 0.163 | 0.135 | 0.125 |
| GP426 | 0.152 | 0.171 | 0.202 | 0.189 | 0.253 | 0.264 | 0.205 |
| GP507 | 0.154 | 0.139 | 0.201 | 0.160 | 0.168 | 0.189 | 0.169 |
| GP582 | 0.167 | 0.147 | 0.180 | 0.285 | 0.290 | 0.324 | 0.232 |

TABLE 8

| HFF2 | 1.000 | 2.000 | 3.000 | 4.000 | 5.000 | 6.000 | Average |
|---|---|---|---|---|---|---|---|
| NCC | 0.184 | 0.228 | 0.228 | 0.219 | 0.223 | 0.237 | |
| Methoxy | 0.203 | 0.233 | 0.237 | 0.247 | 0.227 | 0.256 | 0.234 |
| Ethoxy | 0.204 | 0.269 | 0.259 | 0.245 | 0.299 | 0.249 | 0.254 |
| GP218 | 0.720 | 0.386 | 0.450 | 0.586 | 0.553 | 0.966 | 0.610 |
| GH226 | 0.103 | 0.111 | 0.118 | 0.119 | 0.121 | 0.121 | 0.116 |
| GP426 | 0.178 | 0.235 | 0.233 | 0.249 | 0.228 | 0.225 | 0.225 |
| GP507 | 0.190 | 0.207 | 0.221 | 0.210 | 0.151 | 0.185 | 0.194 |
| GP582 | 0.188 | 0.270 | 0.291 | 0.287 | 0.283 | 0.283 | 0.267 |

TABLE 9

| HFF3 | 1.000 | 2.000 | 3.000 | 4.000 | 5.000 | 6.000 | Average |
|---|---|---|---|---|---|---|---|
| NCC | 0.183 | 0.182 | 0.194 | 0.200 | 0.232 | 0.234 | |
| Methoxy | 0.167 | 0.174 | 0.207 | 0.218 | 0.210 | 0.224 | 0.200 |
| Ethoxy | 0.209 | 0.220 | 0.247 | 0.249 | 0.224 | 0.205 | 0.226 |
| GP218 | 0.083 | 0.100 | 0.098 | 0.081 | 0.110 | 0.103 | 0.096 |
| GP226 | 0.100 | 0.120 | 0.121 | 0.123 | 0.125 | 0.128 | 0.120 |
| GP426 | 0.177 | 0.238 | 0.213 | 0.218 | 0.219 | 0.223 | 0.215 |
| GP507 | 0.150 | 0.237 | 0.198 | 0.179 | 0.205 | 0.199 | 0.195 |
| GP582 | 0.204 | 0.175 | 0.237 | 0.227 | 0.233 | 0.234 | 0.218 |

TABLE 10

| HSF1 | 1 | 2 | 3 | 4 | 5 | 6 | Average |
|---|---|---|---|---|---|---|---|
| NCC | 0.135 | 0.141 | 0.149 | 0.147 | 0.142 | 0.139 | |
| Methoxy | 0.126 | 0.142 | 0.132 | 0.150 | 0.178 | 0.148 | 0.146 |
| Ethoxy | 0.130 | 0.147 | 0.123 | 0.133 | 0.135 | 0.132 | 0.133 |
| GP218 | 0.075 | 0.074 | 0.074 | 0.118 | 0.082 | 0.082 | 0.084 |
| GP226 | 0.086 | 0.092 | 0.091 | 0.092 | 0.095 | 0.093 | 0.092 |
| GP426 | 0.115 | 0.140 | 0.159 | 0.142 | 0.163 | 0.148 | 0.145 |
| GP507 | 0.120 | 0.110 | 0.107 | 0.107 | 0.108 | 0.106 | 0.110 |
| GP582 | 0.150 | 0.132 | 0.150 | 0.118 | 0.149 | 0.138 | 0.140 |

TABLE 11

| HSF2 | 1 | 2 | 3 | 4 | 5 | 6 | Average |
|---|---|---|---|---|---|---|---|
| NCC | 0.156 | 0.142 | 0.179 | 0.170 | 0.140 | 0.170 | |
| Methoxy | 0.173 | 0.192 | 0.153 | 0.220 | 0.189 | 0.187 | 0.186 |
| Ethoxy | 0.179 | 0.219 | 0.206 | 0.203 | 0.165 | 0.194 | 0.194 |
| GP218 | 0.128 | 0.132 | 0.116 | 0.127 | 0.084 | 0.077 | 0.111 |
| GP226 | 0.112 | 0.114 | 0.115 | 0.118 | 0.136 | 0.118 | 0.119 |
| GP426 | 0.174 | 0.152 | 0.149 | 0.219 | 0.187 | 0.212 | 0.182 |
| GP507 | 0.183 | 0.153 | 0.160 | 0.166 | 0.149 | 0.136 | 0.158 |
| GP582 | 0.164 | 0.194 | 0.202 | 0.160 | 0.237 | 0.212 | 0.195 |

1. A composition for comprising
1) a compound of formula (I): 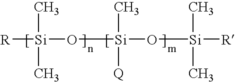
wherein:
m=0-6;
n=6-100;
R and R′ are independently a C₁₋₅ alkyl;
Q is independently selected from the group consisting of OU, VOCH₂CH₃, CH₂CH₃, VOCH₃, OH, O(CH₂)_{y}(OV)_{y}CH₃, (OCH₂CH₂)_{y}OU, (OCH₂CH₂)_{y}OH, VO(CH₂CH₂O)_{y}U, VO(CH₂CH₂O)_{y}V′OH, VOH, VOU, VOV′OU, VCO₂U, CO₂U, VCO₂COU, CO₂H, VCO₂H, COX, VCOX, VCO₂R′, CO₂COU, Aryl, ArylU, ArylUU″, ArylUU′U″, NH₂, VNH₂, NHU, NUU′, NO₂, VNO₂, VCONH₂, CONH₂, VCONHU, CONHU, VCONUU′, CONUU′, halogen, SH, SO₂ and SO₃H;
wherein V and V′ are independently any alkylene, alkenylene or alkynylene group where the number of carbon atoms is between 2 and 31;
wherein U, U′ and U″ are independently any alkyl where the number of carbon atoms is between 1 and 31, or any alkenyl or alkynyl group where the number of carbon atoms is between 2 and 31;
wherein X=halogen;
wherein y=1-100; and
wherein the compound of formula (I) is present in an amount of at least 1% of the composition, and
2) at least one additive selected from the group consisting of a pharmacologically-acceptable carrier, diluent, and excipient.

2. The composition of claim 1 wherein the compound of formula (I) is present in an amount of at least 10% of the composition.

3. The composition of claim 1 wherein the compound of formula (I) is present in an amount of at least 30% of the composition.

4. The composition of claim 1 wherein in the compound of formula (I) n is 17.

5. The composition of claim 1 wherein in the compound of formula (I) m is 1.

6. The composition of claim 1 wherein in the compound of formula (I) R and R′ are C₁ alkyl.

7. The composition of claim 6 wherein Q is VO(CH₂CH₂O)_{y}U.

8. The composition of claim 6 wherein Q is CH₂CH₂CH₂—O—(CH₂CH₂O)_{y}CH₃.

9. The composition of claim 6 wherein Q is CH₂CH₂CH₂—O—(CH₂CH₂O)_{y}CH₂CH₂CH₃.

10. The composition of claim 6 wherein Q is VOH.

11. (canceled)

12. The composition of claim 1 wherein the carrier, diluent or excipient is selected from the group consisting of an oil in water emulsion, a water in oil emulsion, a particulate carrier, a clay, a silicate, a clathrate and other inorganic material.

13. The composition of claim 12 wherein the particulate carrier is talcum powder.

14. The composition of claim 1 further comprising an additive selected from the group consisting of a colourant and a second pharmaceutically active material, wherein the second pharmaceutically active material is not the compound of formula (I).

15. The composition of claim 1 in the form of a gel.

16. The composition of claim 1 in the form of gel sheeting.

17. The composition of claim 1 comprising a low molecular weight poly alcohol that acts as an artificial skin upon drying.

18. The composition of claim 1 wherein the wound is a scar.

19. A treatment method comprising administering an effective amount of a composition to a wound, burn, or other skin condition of a mammal, wherein the composition comprises a compound of formula (I): 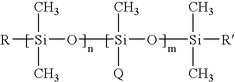
wherein:
m=0-6;
n=6-100;
R and R′ are independently a C₁₋₅ alkyl;
Q is independently selected from the group consisting of OU, VOCH₂CH₃, CH₂CH₃, VOCH₃, OH, O(CH₂)_{y}(OV)_{y}CH₃, (OCH₂CH₂)_{y}OU, (OCH₂CH₂)_{y}OH, VO(CH₂CH₂O)_{y}U, VO(CH₂CH₂O)_{y}V′OH, VOH, VOU, VOV′OU, VCO₂U, CO₂U, VCO₂COU, CO₂H, VCO₂H, COX, VCOX, VCO₂R′, CO₂COU, Aryl, ArylU, ArylUU″, ArylUU′U″, NH₂, VNH₂, NHU, NUU′, NO₂, VNO₂, VCONH₂, CONH₂, VCONHU, CONHU, VCONUU′, CONUU′, halogen, SH, SO₂ and SO₃H;
wherein V and V′ may be independently selected from any alkylene, alkenylene or alkynylene group where the number of carbon atoms is between 2 and 31;
wherein U, U′ and U″ may be independently selected from any alkyl where the number of carbon atoms is between 1 and 31, or any alkenyl or alkynyl group where the number of carbon atoms is between 2 and 31;
wherein X halogen;
wherein y=1-100;
wherein the compound of formula (I) is present in the composition in an amount of at least 1%.

20. A method of treating a wound, burn or other skin condition including the step of administering to a patient a composition, the composition comprising a compound of formula (I): 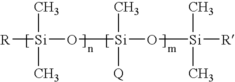
wherein:
m=0-6;
n=6-100;
R and R′ may be independently selected from C₁₋₅ alkyl;
Q may be independently selected from, OU, VOCH₂CH₃, CH₂CH₃, VOCH₃, OH, O(CH₂)_{y}(OV)_{y}CH₃, (OCH₂CH₂)_{y}OU, (OCH₂CH₂)_{y}OH, VO(CH₂CH₂O)_{y}U, VO(CH₂CH₂O)_{y}V′OH, VOH, VOU, VOV′OU, VCO₂U, CO₂U, VCO₂COU, CO₂H, VCO₂H, COX, VCOX, VCO₂R′, CO₂COU, Aryl, ArylU, ArylUU″, ArylUU′U″, NH₂, VNH₂, NHU, NUU′, NO₂, VNO₂, VCONH₂, CONH₂, VCONHU, CONHU, VCONUU′, CONUU′, halogen, SH, SO₂ and SO₃H;
wherein V and V′ may be independently selected from any alkylene, alkenylene or alkynylene group where the number of carbon atoms is between 2 and 31;
wherein U, U′ and U″ may be independently selected from any alkyl where the number of carbon atoms is between 1 and 31, or any alkenyl or alkynyl group where the number of carbon atoms is between 2 and 31;
wherein X=halogen;
wherein y=1-100; and
wherein the compound of formula (I) is present in an amount of at least 1% of the composition; and
wherein said compound migrates through the stratum corneum layer to a lower epidermal skin layer.

21. The method of treating a wound, burn or other skin condition according to claim 20 including the step of administering the composition topically.

22. The method of treating a wound, burn or other skin condition according to claim 21 including the step of administering the composition in an amount effective for remediation of the wound, burn or other skin condition.

23. The method of treating a wound, burn or other skin condition according to claim 20 including the step of administering the composition in an amount effective for treatment of one or more skin condition selected from the group consisting of keloid scarring, hypertrophic scarring, acne scarring, erythematous skin and stretch mark scarring.

24. The method of treating a wound, burn or other skin condition according to claim 20 including the step of administering the composition in an amount effective to modify one or more clinical marker selected from the group consisting of scar colour, scar profile, amount of hair, size of wound and amount of infection.

25. The method of treating a wound, burn or other skin condition according to claim 20 including the step of administering the composition in an amount effective to modify one or more marker selected from the group consisting of number of fibroblasts, alteration of interstitial tissue, epidermal thickness, number of hair follicles and alteration in papillary and reticular dermis.

26. The method of treating a wound, burn or other skin condition according to claim 20 including the step of administering the composition in an amount effective to induce monocyte activation.

27. The method of treating a wound, burn or other skin condition according to claim 20 including the step of administering the composition in an amount effective to suppress fibroblast growth.

28. The method of treating a wound, burn or other skin condition according to claim 20 including the step of administering the composition in an amount effective to down regulate collagen production.

29. The method of treating a wound, burn or other skin condition according to claim 20 including the step of administering the composition in an amount effective to not inhibit cell proliferation.

30. The method of treating a wound, burn or other skin condition according to claim 20 including the step of administering the composition in an amount effective to enhance collagenase activity.
